(19) United States
(12) Patent Application Publication
(10) Pub. No.: US 2015/0216814 A1
(43) Pub. Date: Aug. 6, 2015

(54) DELIVERY OF SMALL INTERFERING RNA AND MICRO RNA THROUGH MEMBRANE-DISRUPTIVE, RESPONSIVE NANOSCALLE HYDROGELS

(71) Applicant: BOARD OF REGENTS, THE UNIVERSITY OF TEXAS SYSTEM, AUSTIN, TX (US)

(72) Inventors: NICHOLAS PEPPAS, AUSTIN, TX (US); WILLIAM LIECHTY, MIDLAND, MI (US)

(21) Appl. No.: 14/685,949

(22) Filed: Apr. 14, 2015

Related U.S. Application Data
(63) Continuation of application No. PCT/US13/64887, filed on Oct. 14, 2013.
(60) Provisional application No. 61/713,610, filed on Oct. 14, 2012.

(51) Int. Cl. A61K 9/51; A61K 31/713

(52) U.S. Cl. CPC A61K 9/5138; A61K 31/713

(57) ABSTRACT

Nanoscale, pH-responsive polycationic networks useful for the delivery of anionic biologic therapeutics and associated methods.

### CROSS-REFERENCE TO RELATED APPLICATION

This application is a continuation of International Application No. PCT/US2013/064887, filed Oct. 14, 2013, which claims the benefit of U.S. Provisional Application Ser. No. 61/713,610 filed on Oct. 14, 2012, which are incorporated by reference.

### STATEMENT OF GOVERNMENT INTEREST

This invention was made with government support under Grant No. CBET 10-33746 awarded by the National Science Foundation. The government has certain rights in the invention.

### BACKGROUND

Polymers have played an integral role in the advancement of drug delivery technology, providing controlled release of therapeutic agents in constant doses over long periods, cyclic dosage, and enabling tunable release of both hydrophilic and hydrophobic drugs. From early beginnings using off-the-shelf materials, the field has grown tremendously, driven in part by the innovations of chemical engineers. Modern advances in drug delivery are now predicated upon the rational design of polymers tailored for specific cargo and engineered to exert distinct biological functions. In particular, hydrogels have been instrumental in the development of polymeric systems for controlled release of therapeutic agents. These materials are attractive for transmucosal and intracellular drug delivery because of their facile synthesis, inherent biocompatibility, tunable physicochemical properties, and capacity to respond to various physiological stimuli.

The landmark discovery of RNA interference (RNAi) in 1998 has sparked a massive research effort in all fields of biological science and redefined our understanding of gene regulation mechanisms. Theoretically, RNAi mediated by small interfering RNA (siRNA) could be used as a powerful and versatile treatment modality to treat nearly any disease resulting from aberrant gene expression. Owing to its remarkable potency and low therapeutic dosage, siRNA holds extraordinary promise as a new biological therapeutic. However, efficient delivery has been implicated as the major hurdle to its widespread clinical application. Although much effort has been directed toward synthetic polymer carriers for siRNA, there remains a paucity of data on the development of oral delivery systems. Nearly all delivery systems undergoing clinical trials (no RNAi therapeutic has achieved FDA approval) rely on naked siRNA, conjugated polymers, or lipid carriers for topical and intravenous administration and do not posses attributes that render them useful delivery vectors for GI targets. It is desirable to develop a novel hydrogel platform for siRNA delivery, a synthetic polymer carrier capable of providing siRNA to disease targets, especially those along the gastrointestinal tract.

### SUMMARY

The present disclosure generally relates to compositions useful in the delivery of anionic therapeutic agents. More particularly, in some embodiments, the present disclosure relates to nanoscale, pH-responsive polycationic networks useful for the delivery of anionic biologic therapeutics and associated methods. The present disclosure provides, according to certain embodiments pH-responsive polycationic networks comprising siRNA in the polymer network. Such siRNA-containing networks may be useful for delivery of siRNA.

In this disclosure, a robust synthesis approach to expand the range of therapeutics currently delivered via hydrogel technology is outlined. Through judicious materials selection and careful design of copolymer composition and molecular architecture, systems capable of responding to distinct physiological cues, with tunable physicochemical properties that are optimized to load, protect, and deliver valuable macromolecular payloads to their intended site of action may be engineered.

The features and advantages of the present invention will be readily apparent to those skilled in the art. While numerous changes may be made by those skilled in the art, such changes are within the spirit of the invention.

### DRAWINGS

Some specific example embodiments of the disclosure may be understood by referring, in part, to the following description and the accompanying drawings.

FIG. 1 is a schematic of pH-responsive hydrogels.

FIG. 2 shows the molecular structure of certain methacrylate monomers and initiator used in synthesis of pH-responsive hydrogels.

FIG. 3 shows representative transmission electron microscopy images of TEGDMA-crosslinked nanogels. PDET (A), PDETB10 (B), PDETB20 (C) PDETB30 (D), PDETBA10 (E), PDETBA10 (F), PDETBA30 (G). Particles stained with uranyl acetate and images collected at 43,000×. Scale bar represents 200 nm.

FIG. 4 shows sample number-average particle size distribution of dry P(DEAEMA-co-TBMA-g-PEGMA) (PDETB30) generated by quantitative particle sizing from TEM micrographs. Distribution mean=52.0, Std Dev=17.3, n=200. Bars represent observed data and line represents best fit Gaussian distribution (R2=0.982).

FIG. 5 shows representative intensity-weighted particle size distribution for P(DEAEMA-co-TBMA-g-PEGMA) crosslinked with 2.5 mol % TEGDMA (PDETB30) in the collapsed (solid) and swollen (dashed) state. Measurements conducted at 25° C. in PBS.

FIG. 6 shows colloidal stability of nanoscale hydrogels. Hydrodynamic diameter (left axis) and polydispersity index (right axis) of P(DEAEMA-co-TBMA-g-PEGMA) networks crosslinked with 2.5 mol % TEGDMA after 4 weeks (filled symbols) and 8 weeks (empty symbols) in aqueous suspension. Data points represent mean of 12 measurements and lines represent a best fit to the data. A hyperbolic tangent fit was applied to the measurements of hydrodynamic diameter and a linear fit was applied to the measurements of polydispersity index.

FIG. 7 shows influence of hydrophobic moiety incorporation on pH-dependent swelling properties in nanoscale hydrogels containing TBMA (A) or TBAEMA (B). Symbols indicate 0 mol % (□), 10 mol % (⋄), 20 mol % (A), or 30 mol % () comonomer based on DEAEMA. Data points represent mean of 12 measurements and lines represent a hyperbolic tangent best fit to the data.

FIG. 8 shows effective surface zeta-potential of polymer formulations synthesized with varying TBMA (A) and TBAEMA (B). Data points represent the mean of 10 measurements±SD.

FIG. 9 shows normalized fluorescent emission spectra of pyrene in 100 mM phosphate buffer and 0.5 mg mL-1 PDETB30 at pH 8.0 (solid) and pH 6.0 (dashed).

FIG. 10 shows influence of t-butyl incorporation on fluorescence emission spectra of nanogels synthesized with TBMA (A) or TBAEMA (B). Nanogels suspended at 0.5 mg mL-1 and pyrene dissolved at 6×10-7 M in 100 mM phosphate buffers. Symbols designate PDET (), PDETB10 (▾), PDETB20 (▪), or PDETB30 (⋄) in (A) and PDET (), PDETBA10 (▾), PDETBA20 (▪), or PDETBA30 (⋄) in (B). Points represent measured data and lines represent best-fit sigmoidal curves.

FIG. 11 influence of hydrophobic moiety on fluorescence emission spectra of pyrene. Pyrene dissolved at 6×10-7 M in 100 mM phosphate buffers with PDET (), PDETB30 (▾), or PDETBA30 (▪) at 0.5 mg mL-1. Points represent measured data and lines represent best-fit sigmoidal curves.

FIG. 12 shows normalized fluorescent excitation spectra of pyrene in 100 mM phosphate buffer and 0.5 mg mL-1 PDET at pH 8.0 (solid) and pH 6.0 (dashed).

FIG. 13 shows influence of TBMA incorporation on pyrene excitation (1338/1333 ratio) in P(DEAEMA-co-TBMA-g-PEGMA) nanogels. Nanogels suspended at 0.5 mg mL-1 and pyrene dissolved at 6×10-7 M in 100 mM phosphate buffers at designated pH values. Symbols designate PDET (), PDETB10 (▾), PDETB20 (▪), or PDETB30 (⋄).

FIG. 14 shows influence of TBAEMA incorporation on pyrene excitation (1338/1333 ratio) in P(DEAEMA-co-TBAEMA-g-PEGMA) nanogels. Nanogels suspended at 0.5 mg mL-1 and pyrene dissolved at 6×10-7 M in 100 mM phosphate buffers at designated pH values. Symbols designate PDET (), PDETB10 (▾), PDETB20 (▪), or PDETB30 (⋄).

FIG. 15 shows influence of hydrophobic moiety on fluorescence excitation spectra of pyrene. Pyrene dissolved at 6×10-7 M in 100 mM phosphate buffers with PDET (), PDETB30 (▾), or PDETBA30 (▪) at 0.5 mg mL-1. Points represent measured data and lines represent best-fit sigmoidal curves.

FIG. 16 shows hemolysis as a function of nanogel concentration and solution pH. Contour plots for PDET, PDETB20 and PDETB30 (top) and PDET, PDETBA20, and PDETBA30 (bottom).

FIG. 17 shows concentration-dependent hemolytic activity of PDET (□), PDETB30 (), and PDETBA30 (□) in 150 mM phosphate buffer at early endosomal pH (pH 6.0). Erythrocytes exposed to various polymer concentrations for 60 min at 37° C. Data points represent the mean of triplicate samples±s.d.

FIG. 18 shows representative time-dependent LDH leakage from Caco-2 cells following 60 min (), 180 min (∘), or 360 min (▾) exposure to PDET. Data points represent the sample mean±std error (n=4). LDH leakage calculated relative to untreated cells and surfactant-lysed cells.

FIG. 19 shows representative time-dependent LDH leakage from Caco-2 cells following 60 min (), 180 min (∘), or 360 min (▾) exposure to PDETB30. Data points represent the sample mean±std error (n=4). LDH leakage calculated relative to untreated cells and surfactant-lysed cells.

FIG. 20 shows representative time-dependent LDH leakage from Caco-2 cells following 60 min (), 180 min (∘), or 360 min (▾) exposure to PDETBA30. Data points represent the sample mean±std error (n=4). LDH leakage calculated relative to untreated cells and surfactant-lysed cells.

FIG. 21 shows polymer-mediated LDH leakage from Caco-2 cells following exposure to PDET (), PDETB10 (▾), PDETB20 (▪), or PDETB30 (⋄) for 60 min (A) or PDET (), PDETBA10 (▾), PDETBA20 (▪), or PDETBA30 (⋄) for 60 min (B).

FIG. 22 shows destabilization of GUV membranes. Intravesicle red fluorescence indicates sucrose-Texas Red. Green fluorescence indicates membrane lipid DHPE-Bodipy FL. GUVs were suspended in 100 mM phosphate buffer at pH 6.5. PDET (A) or PDETB30 (B) in isosmotic phosphate buffer was added at achieve a final concentration of 50 μg ml-1. GUVs after 30 seconds incubation (C and D). Images captured using Zeiss spinning disc confocal microscope at 100×.

FIG. 23 shows cytocompatibility of polycationic nanogels as a function of polymer concentration. Symbols represent PDET (), PDETB20 (▾), PDETB30 (▪), PDETBA20 (⋄), or PDETBA30 (▴). The relative viability of Caco-2 cells was determined by MTS assay following 90 min nanogel exposure and is expressed as a fraction of the control (untreated) cells. Data are expressed as means±SEM, n=8. Lines are to guide the eye. Statistical significance determined via pairwise t-test between cells exposed to PDETB20 and PDET or PDETB30 and PDET (* p<0.005).

FIG. 24 shows cytocompatibility of P(DEAEMA-g-PEGMA) and P(DEAEMA-co-TBMA-g-PEGMA) nanogels as a function of polymer concentration. The relative proliferation of RAW 264.7 cells was determined by MTS assay following 3 h nanogel exposure and is expressed as a fraction of the control (untreated) cells. Data are expressed as means±SEM, n=4. Statistical significance determined via pairwise t-test between cells exposed to PDETB30 or PDET (# p<0.05).

FIG. 25 shows cytocompatibility of PDET and PDETB30 nanogels as a function of polymer concentration following 24 h exposure. The relative proliferation of RAW 264.7 cells was determined by MTS assay and is expressed as a fraction of the control (untreated) cells. Data are expressed as means±SEM, n=4. Statistical significance determined via pairwise t-test between cells exposed to PDET or PDETB30 (# p<0.05).

FIG. 26 shows a schematic degradable nanogel in response to glutathione. Disulfide crosslinks are sensitive to reductive conditions.

FIG. 27 shows reaction scheme for bis(2-methacryloyloxyethyl) disulfide.

FIG. 28 shows pH-responsive behavior of nanogels in suspended in PBS. PDET (□), PDETB30 (◯), and PDESSB30 (♦).

FIG. 29 shows relative proliferation of RAW 264.7 cells upon exposure to PDESSB30 () or PDETB30 (∘) for 360 min. The relative proliferation of RAW cells was determined by MTS assay and is expressed as a fraction of the control (untreated) cells. Data are expressed as mean±SEM, n=4.

FIG. 30 shows light scattering analysis of glutathione-induced degradation. Nanogels dissolved in PBS and exposed to 1 mM (gray) and 10 mM (black) concentrations of glutathione (GSH) and incubated at 37° C.

FIG. 31 shows representative transmission electron microscopy images of PDESSB30 incubated for 2 hours in (A) DI water and (B) 10 mM glutathione solution (left). Particles stained with uranyl acetate and images collected at 26,500× (left) and 20,500× (right).

FIG. 32 shows RNA Loading capacity of poly(DEAEMA-g-PEGMA) (PDET), and poly(DEAEMA-co-BMA-g-PEGMA) (PDETB10), (PDETB20), and (PDETB30) in PBS, pH 5.50. Data represent mean±s.d. (n=3).

FIG. 33 shows RNA loading of poly(DEAEMA-g-PEGMA) (PDET), and poly(DEAEMA-co-BMA-g-PEGMA) (PDETB10), (PDETB20), and (PDETB30) in PBS, pH 5.50 (gray) and in serum-free DMEM, pH 7.40 (white). Polymer and siRNA were combined at a ratio of 10:1 [polymer]:[siRNA]. Fraction of bound RNA determined by Ribogreen assay following 180 min incubation in PBS and 30 min incubation in DMEM. Bars represent the mean±s.d. (n=3).

FIG. 34 shows fluorescent micrographs of PDETB30-OG488 and DY647-siRNA

FIG. 35 shows siRNA delivery to Caco-2 cells as a function of incubation time. Nanogel/Cy3-siRNA complexes were prepared at a 20:1 nanogel/siRNA ratio (g/g) and incubated with cells for designated time points. Data points represent the median fluorescence of live cells as determined by flow cytometry. Dead cells excluded via propidium iodide.

FIG. 36 shows siRNA delivery to Caco-2 cells as a function of nanogel composition and incubation temperature. Nanogel/Cy3-siRNA complexes were prepared at a 20:1 nanogel/siRNA ratio (g/g) and incubated with Caco-2 cells for 60 min. Data points represent the median fluorescence of live cells as determined by flow cytometry. Dead cells excluded via propidium iodide.

FIG. 37 shows fluorescence intensity of RAW 264.7 cells in siRNA delivery experiments. Fluorescence intensity histograms of DY647-siRNA (A) and PDETB30-OG488 (B). Fluorescence histograms generated from live, focused, single cells exposed to PBS (untreated, gray), DY647-siRNA alone (blue), PDETB30-OG488 alone (green), or PDETB30-OG488/DY647-siRNA (red). Data represent the results of two pooled experiments.

FIG. 38 shows DY647-siRNA delivery to RAW 264.7 cells. Nuclear stain (Hoechst 33342) shown in blue, PDETB30-OG488 (Oregon Green 488) shown in green, and DY647-siRNA (DyLight 647) shown in red. Three representative examples of RAW 264.7 cells exposed to DY647-siRNA alone (A-C), PDETB30-OG488 along (D-F), or PDETB30-OG488/DY647-siRNA (G-I) are shown. Images sampled from median region of DY647 histogram. Scale bar represents 7 μm.

FIG. 39 shows fluorescence intensity of Caco-2 cells in siRNA delivery experiments. Fluorescence intensity histograms of DY647-siRNA (A) and PDETB30-OG488 (B). Fluorescence histograms generated from live, focused, single cells exposed to PBS (untreated, gray), DY647-siRNA alone (blue), PDETB30-OG488 alone (green), or PDETB30-OG488/DY647-siRNA (red). Data represent the results of two pooled experiments.

FIG. 40 shows DY647-siRNA delivery to RAW 264.7 cells. Nuclear stain (Hoechst 33342, blue), PDETB30-OG488 (Oregon Green 488, green), and DY647-siRNA (DyLight 647, red) are shown. Three representative examples of RAW 264.7 cells exposed to DY647-siRNA alone (A-C), PDETB30-OG488 alone (D-F), or PDETB30-OG488/DY647-siRNA (G-I) are shown. Images sampled from median region of DY647 histogram. Scale bar represents 7 μm.

FIG. 41 shows GAPDH knockdown in Caco-2 cells following exposure to PDETB30/siRNA. Cells exposed to siRNA-loaded nanogels at designated concentrations of PDETB30 and 250 ng mL-1 (˜20 nM, gray) or 1250 ng mL-1 (˜100 nM, black) GAPDH siRNA. Expression levels measured 48 hrs after transfection. Bars represent the mean of % remaining expression GAPDH expression±s.d. (n=3). * p<0.05, # p<0.01.

FIG. 42 shows GAPDH knockdown in Caco-2 cells following exposure to PDETB30/siRNA or PDESSB30/siRNA. Cells exposed to siRNA-loaded nanogels at 200:1 nanogel:siRNA ratio (g/g, 50 μg mL-1 nanogel and 0.25 μg ml-1 siRNA). Expression levels measured 48 hrs after transfection. Bars represent the mean of % remaining expression GAPDH expression±s.d. (n=3). * p<0.05.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are described in more detail below. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure generally relates to compositions useful in the delivery of anionic therapeutic agents. More particularly, in some embodiments, the present disclosure relates to nanoscale, pH-responsive polycationic networks useful for the delivery of anionic biologic therapeutics and associated methods.

The present disclosure provides, according to certain embodiments pH-responsive polycationic hydrogels formed from a cationic monomer, a hydrophobic moiety (also referred to as hydrophobic monomer), and a crosslinker. Such hydrogels form random copolymers. The pH-responsive polycationic hydrogels undergo a volume phase transition in response to changing pH. The pH-responsive polycationic hydrogels of the present disclosure are methacrylate-based hydrogels. The hydrogels also may comprise PEG molecules at least partially disposed on an exterior surface of the hydrogel. The pH-responsive polycationic hydrogels are capable of swelling and deswelling in response to a change in pH. Accordingly, such hydrogels may be used to deliver, and my further comprise, anionic therapeutics such as, for example, anionic biologics liked siRNA.

In operation, an anionic therapeutic may be included within the polymer network of the pH-responsive polycationic hydrogels, which also may be capable of enhancing cellular internalization. For example, at physiological pH (˜7.4) a pH-responsive polycationic hydrogel may exist in a collapsed state thereby trapping an anionic therapeutic within a polymer network. When the pH-responsive polycationic hydrogel is introduced into a lower pH environment, such as, for example, within an endosome of a cell, the polymer network swells allowing release of the anionic therapeutic (e.g., siRNA, microRNA, and DNA).

In certain embodiments, the pH-responsive polycationic hydrogels of the present disclosure are cytocompatiable (e.g., >80% at 100 ug/mL), have a size suitable for cellular delivery (e.g., 20-200 nm), are capable of binding nucleic acids (e.g., RNA binding >5 wt %), have a positive surface charge at pH˜7.4, have pH response that is tunable (e.g., collapsed at pH 7.4 and swollen at pH 5.5-6.5), and lower cell membrane disruption at pH˜7.4 and higher cell membrane disruption at pH<˜7.

In general, suitable cationic monomers contain ionizable tertiary amine groups. By way of explanation, protonation of the tertiary amine group causes swelling by recruiting mobile counterions and increasing osmotic pressure in the hydrogel; and, electrostatic repulsion of neighboring amine groups also contributes to this volume phase transition. Examples of suitable cationic monomers include tertiary amino methacrylates, dimethyl amino ethyl methacrolyates, diethyl amino ethyl methacrolyates, diisopropyl amino ethyl methacrolyates, morpholino ethyl methacrylates, polylysine methacrylates. Other suitable cationic monomers include 2-(diethylamino)ethyl methacrylate (DEAEMA) and 2-(tert-butylamino)ethyl methacrylate (BAEMA). The cationic content must be optimized to permit binding of anionic biomolecules (e.g. siRNA, miRNA) while avoiding undue toxicity to excess cationic content. The cationic monomers will typically comprise between about 50 and about 80 mol % of the hydrogel formulation.

Excess cationic content in polymer delivery systems can have deleterious effects. High cationic charge density is frequently correlated with toxicity. Accordingly, the hydrogels of the present disclosure also include a hydrophobic moiety (e.g., to improve cytocompatibility and polymer-induced membrane destabilization). The hydrophobic moiety modulates the physiochemical properties of the hydrogel by altering the relative strength between polymer-polymer interactions and polymer-solvent-ion interactions. In this fashion, increasing hydrophobic content of pH-responsive polycationic hydrogels increases the strength of polymer-polymer interactions and favors a deswollen (collapsed) conformation. A consequence of this effect is a requisite increase in the ionization energy (e.g. lower pH) required to favor polymer-solvent-ion interactions and induce osmotic swelling of the polycationic hydrogel. As hydrophobic content increases, greater proton activity or greater ionization (i.e. lower pH) is required to promote polymer/solvent/ion interaction over polymer/polymer interaction. As expected, this effect also leads to a decrease in the onset of pH-dependent gel swelling. Therefore, the chemical nature and composition of the hydrophobic moiety may be tailored to tune the pH response of the pH-responsive polycationic hydrogels.

The critical pH required to induce a pH-dependent, hydrophobic to hydrophilic transition can be tuned according to the type and composition of hydrophobic moiety in the nanoscale hydrogels. Hydrophobic comonomers in the hydrogel serve to promote polymer-polymer interactions and decrease the critical swelling pH necessary for osmotic gel swelling. These polycationic hydrogels are able to destabilize biological membranes most efficiently at or near their critical swelling pH. Hydrophobic moieties are used to match the hydrogel critical swelling pH with endosomal pH (pH˜6.5-7.0) to facilitate endsomal escape of the encapsulate cargo. Additionally, increasing hydrophobic moiety concentration in the polycationic hydrogel decreases cationic charge density. Cationic charge density is commonly associated with cellular toxicity, an unacceptable property in polymeric drug delivery systems. In these polycationic hydrogels, decreasing charge density leads to reduced nonspecific toxicity in model cell lines.

Any hydrophobic moiety capable of copolymerizing with the cationic polymer may be suitable. In some embodiments, the cationic polymer and hydrophobic moiety form a copolymer. Examples of suitable hydrophobic moieties include reactive methacrylates and acrylates, including aliphatic (meth)acrylates such as, for example, propyl (meth)acrylate, tert-butyl (meth)acrylates, 2-(tert-Butylamino)ethyl methacrylate, n-butyl (meth)acrylates, phenyl (meth)acrylates, iso-butyl (meth)acrylates, hexyl (meth)acrylates, iso-decyl (meth)acrylates, and lauryl (meth)acrylates. The amount of hydrophobic moiety should be sufficient to decrease critical swelling pH while permitting hydrogel ionization and volume phase transition. In general, the amount of hydrophobic moiety will be from about 20% to about 50 mol %. In certain embodiments, the amount of hydrophobic moiety will be 20, 25, 30, 35, 40, 45, or 50 mol %.

In general the amount of cationic monomer and hydrophobic moiety may be adjusted to achieve the desired properties. By way of explanation, the inclusion of progressively higher amounts of hydrophobic moiety shifts the hydrophobic-hydrophilic transition downward to lower pH values. This is because a more hydrophobic network will experience higher van der Waals forces and will consequently require greater ionization energy (in the form of more protons) to induce a phase conformation. In general, suitable ratios of cationic monomer to hydrophobic moiety are from about 20% to about 50%. In certain embodiments, the ratios of cationic monomer to hydrophobic moiety is 20%, 25%, 30%, 35%, 40%, 45%, or 50%.

The pH-responsive polycationic hydrogels also include a crosslinker. The crosslinker helps create the polymer network by connecting polymer chains through covalent bonds. Such crosslinking also provides mechanical integrity to the resultant hydrogels. In general, suitable crosslinkers are capable of covalently linking polycationic polymers. Suitable crosslinkers may be homobifunctional (both ends are same) methacrylate crosslinkers such as, for example, ethylene glycol dimethacrylate (EGDMA), tetra(ethylene glycol) dimethacrylate (TEGDMA), and poly(ethylene glycol) dimethacrylate (PEGDMA), or combinations thereof.

The amount of crosslinker or density of crosslinking may vary depending on the average pore size desired. Increasing the crosslinking density results in hydrogels with smaller average pore sizes. Thus, the average pore size may be optimized for a particular anionic biologic therapeutics to be delivered. In general, the average pore size should be large enough to permit delivery of a chosen anionic biologic therapeutic, but small enough to prevent substantial diffusion of the chosen anionic biologic therapeutics out of the hydrogel when the hydrogel is in a collapsed state. In general, the amount of crosslinker may range from about 0.5 to 5 mol % of the hydrogel. In certain embodiments, the crosslinker is 0.5, 1, 2, 3, 4, or 5 mol %.

In certain embodiments, the crosslinker may be degradable and thereby provide pH-responsive polycationic hydrogels that are degradable. In such embodiments, the crosslinker is at least partially disrupted (e.g., covalent bonds broken) by conditions within a cell. For example, the crosslinker may be chemically degraded by enzymes present within the cell. Examples of suitable degradable crosslinkers include, homobifunctional disulfide crosslinkers such as, for example, bis(2-methacryloyloxyethyl) disulfide (SSXL).

In certain embodiments, the pH-responsive polycationic hydrogels may have a size suitable for delivery into a cell. In certain embodiments, the pH-responsive polycationic hydrogels may have a z-average particle size diameter from about 20 nm to about 200 nm. In certain embodiments, the pH-responsive polycationic hydrogels may have a z-average particle size diameter from about 90 nm to about 100 nm. In specific embodiments, the pH-responsive polycationic hydrogels in a collapsed state may have a z-average particle size diameter from about 20 nm to about 100 nm. In specific embodiments, the pH-responsive polycationic hydrogels in a swollen state may have a z-average particle size diameter from about 100 nm to about 200 nm. In certain embodiments, the dry hydrogel has a number-average particle size diameter of from about 40 nm to about 80 nm. In certain specific embodiments, the dry hydrogel has a number-average particle size diameter of from about 50 nm.

As noted above, the pH-responsive polycationic hydrogels of the present disclosure also may comprise poly(ethylene glycol) (PEG) or polyoxazoline (POZ) polymers at least partially disposed on an exterior surface of the hydrogel. By way of explanation, PEG may provide improved biocompatibility to the hydrogel, as well as colloidal stability. The PEG is covalently attached to the cationic polymer's backbone. Suitable PEG/POZ include those having a molecular weight of from 1,000 Da to 10,000 Da; for example, 1,000-5,000 Da, 5,000-8,000 Da, 8,000-10,000 Da. Examples of suitable PEG molecules include, but are not limited to PEG having functional anhydride esters, heterobifunctional PEG, poly(ethylene glycol) methyl ether methacrylate (PEGMA), and polyoxazoline polymers with methyl (PMOZ), ethyl (PEOZ), and propyl (PPOZ) pendant groups, or combinations thereof.

In one embodiment, the pH-responsive polycationic hydrogel is P(DEAEMA-co-TBAEMA-g-PEGMA), where PEGMA is poly(ethylene glycol) methyl ether methacrylate, DEAEMA is 2-(diethylamino) ethyl methacrylate, and TBAEMA is 2-(tert-butylamino)ethyl methacrylate.

In one embodiment, the pH-responsive polycationic hydrogel is P(DEAEMA-co-TMBA-g-PEGMA), where TMBA is tert-butyl methacrylate.

The pH-responsive polycationic hydrogels of the present disclosure may be synthesized via UV-initiated, oil-in-water photoemulsion polymerization.

In certain embodiments, the pH-responsive polycationic hydrogels provide a protective encapsulation and the mechanical integrity and chemical stability required to facilitate local delivery to a target site in the gastrointestinal tract. In such cases, the change in pH may arise, for example, from exposure to gastric fluids such as stomach or intestinal fluids. In certain embodiments, the polycationic networks are specifically designed for delivery to disease sites along the gastrointestinal tract, with potential utility in Crohn's disease, ulcerative colitis, celiac disease, and gastrointestinal carcinomas. Other potential uses for this technology encompass any biological therapeutic possessing a slight negative charge. This includes, but is not limited to proteins, plasmid DNA, microRNA, and short hairpin RNA.

To facilitate a better understanding of the present invention, the following examples of certain aspects of some embodiments are given. In no way should the following examples be read to limit, or define, the entire scope of the invention.

### EXAMPLES

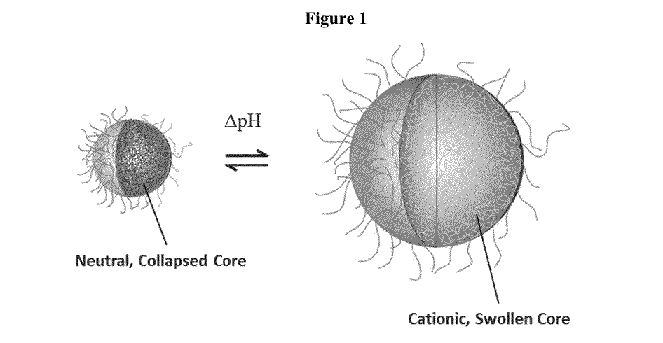

Tunable, responsive nanogels containing tert-butyl methacrylate and 2-(tert-butylamino)ethyl methacrylate. In the present study, we examine the influence of TMBA and TBAEMA on the aqueous solution properties of poly(ethylene glycol) methyl ether methacrylate (PEGMA)-grafted DEAEMA nanogels. This represents, to the best of our knowledge, the first known example of P(DEAEMA-co-TBAEMA-g-PEGMA) nanogels synthesized via aqueous photoemulsion polymerization. We also provide a direct comparison of physicochemical properties with a P(DEAEMA-co-TMBA-g-PEGMA) nanogel system. This polymerization method represents a platform from which abundant combinations of methacrylate-based hydrogels could be produced to create responsive hydrogels with nanoscale dimensions and tunable physicochemical properties (FIG. 1).

Nanogel Synthesis.

Hydrogel particles of nanoscale dimensions were synthesized via UV-initiated free radical photoemulsion polymerization/crosslinking Briefly, DEAEMA, TBAEMA, TBMA, and TEGDMA were passed through a column of basic alumina powder to remove inhibitor prior to use. PEGMA was used as received. DEAEMA, TEGDMA, and TBMA or TBAEMA were added to an aqueous solution containing 5 wt % PEGMA, Irgacure 2959 (Ciba Geigy, Tarrytown, N.Y.) at 0.5 wt % of total monomer, 4 mg mL⁻¹ Brij-30 and 1.16 mg mL⁻¹ (3.4 mM) ionic surfactant MyTAB. The reaction pH was routinely pH 8.5. The mixture was emulsified using a Misonix Ultrasonicator (Misonix, Inc., Newtown, Conn.). The emulsion was purged with nitrogen gas and exposed to a UV source for 2.5 hr with constant stirring. Reagent quantities are tabulated in Table 1.

TABLE 1
Reagents used for nanoscale hydrogel synthesis

| | | mol t- | | | | | | | Monomer |
|---|---|---|---|---|---|---|---|---|---|
| | | butyl/100 mol | DEAEMA | PEGMA | TBMA | TBAEMA | TEGDMA | Water | Fraction in |
| Name | Polymer | DEAEMA | (g) | (g) | (g) | (g) | (g) | (mL) | Water (w/v) |
| PDET | P(DEAEMA-g-PEGMA) | 0 | 2.500 | 5.000 | — | — | 0.121 | 50 | 0.1 |
| PDETB10 | P(DEAEMA-co-TBMA- | 10 | 2.322 | 5.000 | 0.178 | — | 0.124 | 50 | 0.1 |
| | g-PEGMA) | | | | | | | | |
| PDETB20 | P(DEAEMA-co-TBMA- | 20 | 2.168 | 5.000 | 0.332 | — | 0.126 | 50 | 0.1 |
| | g-PEGMA) | | | | | | | | |
| PDETB30 | P(DEAEMA-co-TBMA- | 30 | 2.033 | 5.000 | 0.467 | — | 0.128 | 50 | 0.1 |
| | g-PEGMA) | | | | | | | | |
| PDETBA10 | P(DEAEMA-co- | 10 | 2.273 | 5.000 | — | 0.227 | 0.121 | 50 | 0.1 |
| | TBAEMA-g-PEGMA) | | | | | | | | |
| PDETBA20 | P(DEAEMA-co- | 20 | 2.084 | 5.000 | — | 0.416 | 0.121 | 50 | 0.1 |
| | TBAEMA-g-PEGMA) | | | | | | | | |
| PDETBA30 | P(DEAEMA-co- | 30 | 1.924 | 5.000 | — | 0.576 | 0.121 | 50 | 0.1 |
| | TBAEMA-g-PEGMA) | | | | | | | | |

Nanogel Purification.

MyTAB, Brij 30, and unreacted monomers were removed by repeatedly inducing polymer-ionomer collapse, separating particles by centrifugation for 10 min at 3,200×g, and resuspending in 0.5 N HCl. To monitor the progress of surfactant removal and polymer purification, aliquots of the supernatant from each purification cycle were frozen, lyophilized, and dissolved in DMSO-d₆ for ¹H-NMR analysis. Polymer particles were dialyzed against ddH₂O for at least 7 days in 12-14 kDa molecular weight cutoff dialysis tubing (Spectrum Labs, Rancho Dominguez, Calif.) with water changes twice daily. Following dialysis, polymers were flash frozen in liquid N₂ and lyophilized for 5 days.

¹H-NMR Spectroscopy.

The composition of uncrosslinked polymer formulations and purification fractions were investigated using a Varian (Palo Alto, Calif.) DirectDrive 400 MHz nuclear magnetic resonance spectrometer equipped with automatic sampler. All glassware, including NMR Tubes (Wilmad Lab Glass, Vineland, N.J.), 2 mL sample vials, and Pasteur pipettes were dried overnight in a vacuum oven. Uncrosslinked polymer samples of approximately 50 mg were weighed directly in sample vials and D₂O was added to bring the final polymer concentration to 25 mg mL⁻¹. Samples were briefly sonicated in a sonic bath and transferred to NMR tubes for subsequent analysis. Aliquots of the purification supernatants were frozen at −80° C. and lyophilized with a Labconco FreeZone −105° C. benchtop lyophilizer (Kansas City, Mo.) for 48 hours. The resulting dry residue was dissolved in 0.75 ml deuterated DMSO and transferred to NMR tubes. All NMR Spectra were analyzed using SpinWorks 3™ software.

Nanogel Characterization

Transmission Electron Microscopy.

Transmission electron micrographs were collected using a FEI Tecnai (Hillsboro, Oreg.) Transmission Electron Microscope (80 kV) at magnifications from 16,500× to 160,000×. Lyophilized particles were diluted in ddH₂O and stained with 2% uranyl acetate immediately prior to imaging. Typically, 5 μL of 0.02 w/v % of particle suspension was dropped onto a 400-mesh Formvar-coated copper TEM grid (Electron Microscopy Sciences, Hatfield, Pa.) and excess liquid wicked off using filter paper. An equivalent volume of 2 w/v % uranyl acetate was then added to the grid and allowed to stain for 60 seconds before excess liquid was wicked away with filter paper. Particle volume in the dry state was taken as the cube of mean diameter from TEM images. Particle diameters were calculated from the particle area as determined by an ImageJ particle sizing algorithm. Reported values represent the mean±standard deviation (n>50).

Dynamic Light Scattering Analysis.

The intensity-average hydrodynamic diameter of nanogels in aqueous suspension was measured using a measured using a Brookhaven ZetaPlus instrument (Brookhaven Instruments, Holtsville, N.Y.) operating with a 659 nm diode laser source. Dynamic light scattering (DLS) measurements of particle size and its response to dynamic pH were conducted by resuspending lyophilized particles in PBS at 0.75 mg mL⁻¹. The suspension pH was adjusted to 10.5 using 1 N NaOH and gradually lowered to pH 3.5 using 1 N HCl. Measurements of the z-average particle size were collected at 23° C. Subsequent studies employed a Malvern Zetasizer NanoZS (Malvern Instruments Corp., Malvern, UK) operating with a 633 nm laser source equipped with MPT-2 Autotitrator. DLS measurements of particle size and pH-responsive behavior were conducted by resuspending lyophilized particles in PBS at 0.5 mg mL⁻¹. The suspension pH was adjusted to 10.5 using 1 N NaOH and gradually lowered to pH 3.5 using 1 N HCl. Measurements of the z-average particle size were collected at 25° C. and pH intervals of 0.5.

Titration of Nanogel Suspensions.

The pKa values for nanogel suspensions were measured using a Malvern MPT-2 Autotitrator. Nanogels were equilibrated at 1 mg ml⁻¹ in 100 mM NaCl for 24 h before use. The solution pH was adjusted to pH 3.0 with standardized 0.1 M HCl. The solution pH was slowly raised with to pH 11.0 with increments of standardized 0.1 M NaOH. The solution pH was recorded after each NaOH addition. Nanogel pKa values were estimated as the inflection point of the titration curve. All measurements were conducted at 20° C.

Fluorescence Spectroscopy.

Pyrene (Puriss grade, >99.0%, Sigma-Aldrich, St. Louis, Mo.) was used without further purification. Disodium phosphate heptahydrate (Na₂HPO₄.7H₂O) and sodium phosphate monohydrate (NaH₂PO₄.H₂O) were purchased from Fisher Chemical. Phosphate buffer solutions from pH 5.8-pH 8.0 were prepared by combining solutions of 0.2M NaH₂PO₄.H₂O and 0.2M Na₂HPO₄.7H₂O. Polymer solutions were prepared by suspending dry nanoparticles in ultrapure DI water at a concentration of 1 mg mL⁻¹. These preceding two solutions were then mixed in equal volumes to give a final concentration of nanoparticles at 0.5 mg mL⁻¹ in 100 mM phosphate buffer. Pyrene was dissolved in methanol at 1 mM. Fluorescence spectra were collected on a Fluorlog-3 Spectrofluorometer (Jobin Yvon, Horiba Scientific, Edison, N.J.). Emission spectra were collected with λₑₓ=339 nm, 1 nm increments, 1.5 nm slit with for excitation, 1 nm slit width for emission, and 0.8 s integration time. Excitation spectra were collected with λₑₘ=390 nm, 1 nm increments, 1 nm slit for excitation, and 1.5 nm for emission, and 0.8 s integration time.

Electrophoretic Light Scattering.

The effective surface ξ-potential of the polymer networks was measured using a Brookhaven ZetaPlus instrument (Brookhaven Instruments Corp.) operating with a 659 nm diode laser source. Measurements of ξ-potential as a function of pH were conducted by resuspending lyophilized particles in 5 mM phosphate buffer at 0.5 mg mL⁻¹. The suspension pH was adjusted to 10.5 using 1 N NaOH and gradually lowered to pH 3.5 using 1 N HCl. Electrophoretic light scattering measurements of the surface ξ-potential were collected at 22° C. with nanogels suspended in 5 mM sodium phosphate.

Results and Discussion

Nanogel Synthesis.

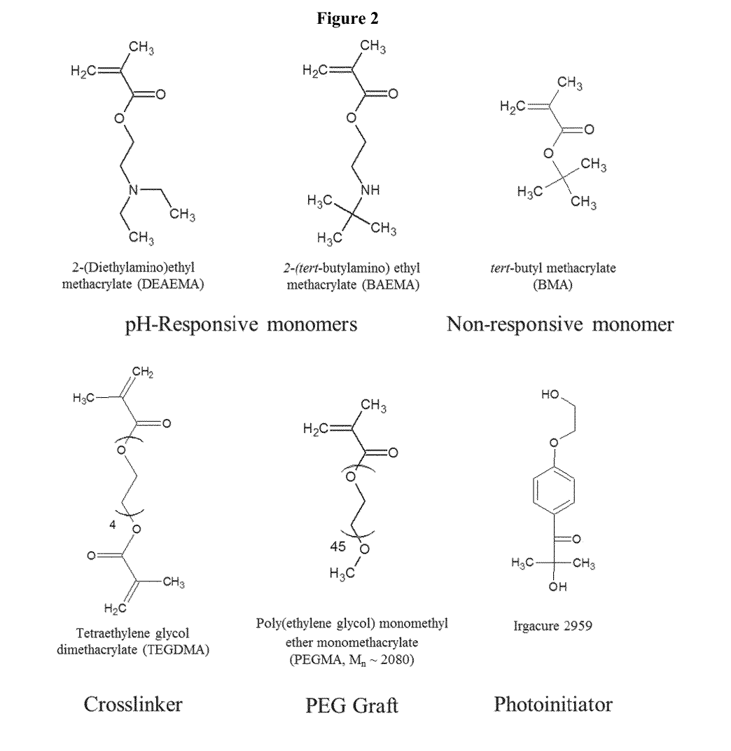

A series of tunable, polycationic nanoscale hydrogels comprised of a crosslinked core of PDEAEMA surface grafted with PEG was synthesized using photoemulsion polymerization. Polymer composition was varied from 0˜25 mol % TBMA or TBAEMA in the copolymer to determine the effect of core hydrophobicity on physicochemical properties. A methoxy-terminated poly(ethylene glycol) methacrylate (PEGMA, MW˜2080) was employed as an emulsion stabilizer and to provide grafted PEG chains on the nanogel surface. PEGMA is commercially available and routinely used as a reactive stabilizer in the aqueous emulsion polymerization of methacrylate copolymers. The MW 2080 PEGMA was chosen because previous work has indicated a minimum PEG graft size of 2 kDa was needed to minimize nonspecific protein adsorption. Monomers used in this synthesis are seen in FIG. 2.

As this polymerization method is a UV-initiated free radical polymerization, the copolymer could be optimally assumed to be random, whereby monomers are incorporated into the copolymer based on their relative feed concentrations and reactivities in no preferential order. This assumption can be evaluated by examining the reactivity ratios (r₁, r₂) for constituent monomer pairs (M₁ and M₂). Generally, copolymerizations with pairwise reactivity ratio products near one (r₁r₂=0.5-2) can be considered to obey moderate ideal polymerization behavior. To estimate the reactivity ratios between methacrylate monomer pairs, the Q-e scheme was used to calculate reactivity ratio (r₁ and r₂) values for constituent monomer pairs (M₁ and M₂). Reported Q-e values and resulting reactivity ratios are tabulated in Table 2.

TABLE 2
Reactivity data of comonomers in photoemulsion polymerization for
fixed M₁ of DEAEMA

| | | Q [symbol] | e [symbol] | r₁ | r₂ | r₁r₂ |
|---|---|---|---|---|---|---|
| M2 | DEAEMA | 2.08 | 0.42 | 1.0 | 1.0 | 1.0 |
| | TBMA | 1.18 | −0.35 | 1.28 | 0.43 | 0.55 |
| | TBAEMA | 0.98 | 0.17 | 1.91 | 0.49 | 0.94 |

[symbol]

Tabulated data

From these data, it can be seen that all estimated values for r₁ and r₂ lie between 0.4-2.0, suggesting that the resultant copolymer can be described as ideal, with copolymer compositions closely related to the feed concentration. For the purpose of this analysis, the ratio between hydrophobic (TBMA or TBAEMA) and ionizable (DEAEMA) monomer was considered to be the key ratio in modulating network hydrophobicity and its influence on resultant physicochemical properties, such as dynamic swelling/deswelling, zeta potential, etc. It is well known that adjusting polymer composition and altering polymer-solvent interactions can impact various critical phenomena, including temperature (LCST/UCST) or pH at which polymer chains undergo phase transitions. For this reason, polymer formulation nomenclature was established such that the numerical suffix on the polymer name (e.g. PDETB30 or PDETBA20) refers to the moles of hydrophobic monomer (TBMA or TBAEMA) per 100 moles of DEAEMA.

Nanogel Purification.

Purification was achieved by repeatedly inducing a polyelectrolyte-ionomer transition. Following polymerization, 1 N HCl was added directly to the reaction flask at 1:1 (vol/vol) ratio with the reaction mixture to protonate DEAEMA pendant groups. Following this step, the acidified reaction mixture was added to acetone to bring the final acetone concentration to 80 vol %. The addition to an organic solvent was used to lower the dielectric strength (c) of the suspension and facilitate the transition from polyelectrolyte regime to ionomer regime. In the polyelectrolyte regime, counterions diffuse freely through polymer networks, while in the ionomer regime, counterions are bound to ionized groups on the polymer network in ion pairs. The energy gain (E) from electrostatic attraction of ions can be described by the equation E=e²/(∈a), where e is the charge of the ion species, ∈ is the dielectric strength, and a is the distance between charges in the ion pair.

The ionomer regime is rare in solvents with a sufficiently high dielectric constant (e.g. water, c=81). As the solvent dielectric strength is lowered, however, the formation of ion pairs decreases the concentration of mobile counterions and concomitantly the gel osmotic pressure. Moreover, these ion pairs attract one another through dipole-dipole interactions and form multiplets. These multiplets act as additional physical crosslinks in the gel and support gel collapse. By using the polymer-ionomer transition, these nanogels can be simultaneously collapsed and ionized, a state that resulted in rapid flocculation and sedimentation. The ionomer phase was then separated from the solvent by centrifugation. Surfactant removal was monitored by analyzing the supernatant from each purification cycle via ¹H-NMR. A purification cycle is defined as (1) protonation of DEAEMA with 0.5 N HCl, (2) ionomer phase transition by addition of 4 vol. equivalents of acetone (80 vol % acetone total), and (3) centrifugation of ionomer/solvent mixture.

The uncrosslinked polymer chains were purified in a similar fashion, albeit with much higher relative centrifugal force (RCF). Sedimentation of uncrosslinked polymer chains following polyelectrolyte-ionomer transition required RCF of 30,000×g or greater. The absence of an oxyethylene peak (δ=3.6 ppm) in the polymer synthesized without PEGMA, P(DEAEMA-co-TBMA) (PDB30), serves as additional evidence that Brij-30 was successfully removed during the purification process.

Determination of Polymer Composition.

To verify copolymer composition, the ¹H NMR spectra of uncrosslinked nanoscale hydrogels were collected and analyzed. The results are summarized in Table 4. The resultant copolymer ratio of t-butyl (either from TBMA or TBAEMA) to DEAEMA closely mirrors that of the comonomer ratio in the feed. This result is expected given the reactivity ratios of constituent comonomers, all containing methacrylate groups, are relatively similar, as previously shown in Table 2. Furthermore, the pairwise products of reactivity ratios (r₁r₂) lie within the region described by ideal radical polymerizations, resulting in a random copolymer.

TABLE 4
Polymer composition as determined by ¹H-NMR for copolymers containing quantities
of t-butyl methacrylate (TBMA) or t-butylaminoethyl methacrylate (TBAEMA).

| | | | t-butyl (TBMA or | | | | | |
|---|---|---|---|---|---|---|---|---|
| | | | TBAEMA) | | DEAEMA | | PEGMA | |
| | | mol 1-butyl/100 mol | mol % in | mol % in | mol % in | mol % in | mol % in | mol % in |
| Name | Polymer | DEAEMA | feed | polymer^{a} | feed | polymer^{a} | feed | polymer^{a} |
| PDE | P(DEAEMA-g- | 0 | — | — | 91.8 | 99.5 | 8.2 | 0.48 |
| | PEGMA) | | | | | | | |
| PDB30 | P(DEAEMA-co- | 30 | 23.1 | 24.6 | 76.9 | 75.4 | — | — |
| | TBMA) | | | | | | | |
| PDEB30 | P(DEAEMA-co- | 30 | 21.6 | 21.4 | 72 | 78.1 | 6.4 | 0.45 |
| | TBMA-g-PEGMA) | | | | | | | |
| PDEBA20 | P(DEAEMA-co- | 20 | 15.5 | 13.5 | 77.6 | 86.1 | 6.9 | 0.38 |
| | TBAEMA-g-PEGMA) | | | | | | | |

^{a}Determined by ¹H-NMR

The peak assignments and spectra for both TBMA- and TBAEMA-containing copolymers was performed. For this analysis, the [—N—(CH₂—CH₃)₂—] (peak d, 3.1 ppm) was selected as the basis for determining the relative molar quantity of DEAEMA, the [—C—(CH₃)₃—] (peak h, 1.3 ppm) for determining the relative molar quantities of TBMA and TBAEMA, and the [—O—CH₂—CH₂—] (peak f, 3.6 ppm) for determining the relative molar quantity of PEGMA.

A notable observation of the data in Table 4 is the poor efficiency with which PEGMA was incorporated into the copolymer; below 10% of PEGMA in the feed was detected in the copolymer. PEGMA is a large (MW˜2,080) and highly hydrophilic macromonomer and consequently may not partition into the oil phase of the emulsion, where DEAEMA, TBMA, and TBAEMA reside. Rather, it is far more likely that the PEG macromonomer adsorbed to the oil droplet surface along with Brij®-30 and MyTAB, serving to reduce interfacial tension at the oil-water boundary. Based on the relative molar compositions determined in Table 4, the copolymer contains approximately 5 wt % PEG.

A consequence of the PEG macromonomer's preferential association with the emulsion interface and water phase is that it will predominantly graft to the particle surface, with incorporated PEG chains protruding in the surrounding aqueous media. Indeed, ¹H-NMR studies of crosslinked nanogels (FIG. S-1) revealed a strong peak at 3.6 ppm (peak a), indicative of oxyethylene protons from PEG. Protons from the ethlyamino groups of DEAEMA (peak c, 3.2 ppm and peak d, 1.25 ppm) are also present, although suppressed relative to their uncrosslinked spectra. These results suggest that the vast majority of DEAEMA and TBMA/TBAEMA protons are sequestered in the network core, unavailable for solvation and detection by ¹H-NMR. This PEG coating may serve to impart colloidal stability and minimize opsonization potential in biomedical applications.

Determination of Nanogel Size

Transmission Electron Microscopy.

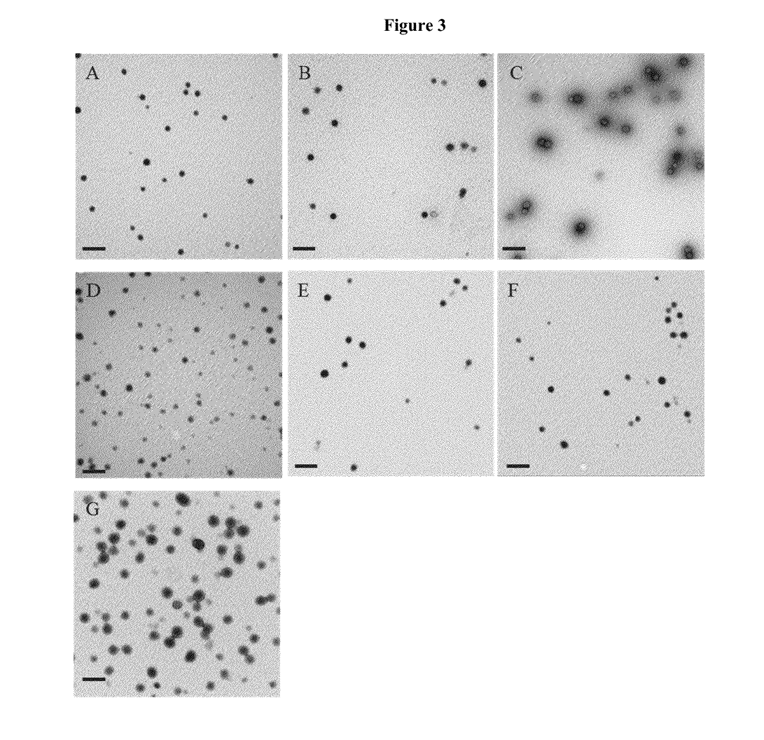

Nanoscale hydrogels of varying composition were initially subjected to TEM measurements to confirm nanoscale dimensions. Analysis of TEM micrographs revealed successful formation of nanoscale hydrogel networks. Nearly all preparations appear to have a narrow particle size distribution with a mean diameter of approximately 50 nm, as seen in FIG. 3. Diameters of the dry nanogel formulations are tabulated in Table 5.

TABLE 5
Calculated diameters of dry nanogels from TEM micrographs. Reported
values represent the mean ± s.d. (n > 150).

| Name | Calculated diameter (nm) |
|---|---|
| PDET | 47 ± 13 |
| PDETB10 | 60 ± 26 |
| PDETB20 | 50 ± 22 |
| PDETB30 | 52 ± 17 |
| PDETBA10 | 50 ± 18 |
| PDETBA20 | 63 ± 21 |
| PDETBA30 | 66 ± 25 |

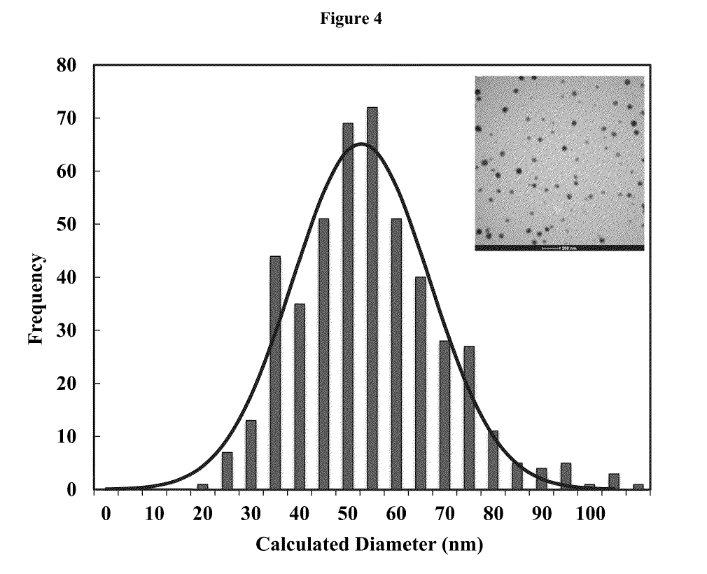

The particle area was determined using ImageJ software to identify particles based on relative contrast between particle and background. This measurement was then used to obtain the dry diameter of nanoscale hydrogels. Images obtained at 26,500× and 43,000× magnification were used most frequently to construct the number-average particle size distribution, as they offered to best combination of particle number, typically 40-50 particles/image, and resolution. In practice, however, images obtained at magnifications of 16,500×-60,000× could be used with little variation in the calculated diameter and standard deviation. Both PDETBA20 (FIG. 3, Panel F) and PDETBA30 (FIG. 3, Panel G) exhibit a mean particle size greater than that of the other formulations, 63 nm and 66 nm. This can perhaps be ascribed to the staining procedure. A staining time of 1 minute was determined sufficient for uranyl acetate to penetrate nanogels of TEGDMA-crosslinked P(DEAEMA-co-TBMA-g-PEGMA) (PDETB30), providing homogenous staining and high contrast. However, this staining time was not sufficient for copolymers with 20 mol % TBAEMA (PDETBA20) and 30 mol % TBMA (PDETBA30) and is most evident in FIG. 3, Panel G, where a hazy ring outlines the particle perimeter. This is present, albeit more subtly, in FIG. 3, Panel F. This blurred boundary made identification of particle perimeter more inaccurate and likely resulted in an overestimation of the true particle area. In all cases, the number-average particle size distribution was roughly Gaussian, an example of which is seen in FIG. 4.

Dynamic Light Scattering.

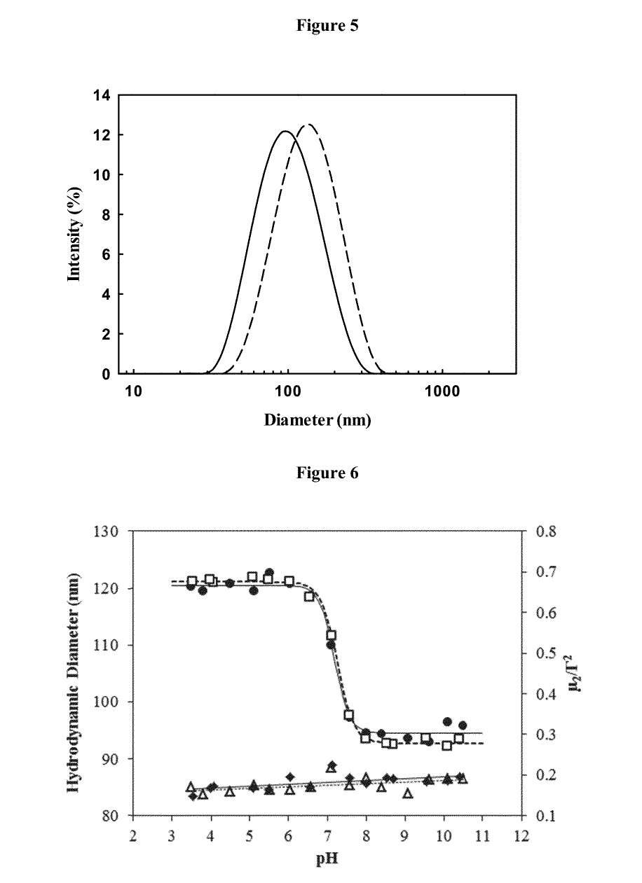

Dynamic light scattering was used to investigate hydrodynamic diameter of the nanogel suspensions in aqueous solutions. The hydrodynamic diameter and polydispersity index reported represent those determined by cumulant analysis as outlined in ISO 13321. Other analysis algorithms exist, such as CONTIN and NNLS, but the cumulant analysis is the readily accepted and has been widely adopted by industry and academia. The limiting assumption of this method is a single distribution of spherical particles. The method returns a single z-average particle size, which represents a hydrodynamic diameter based on the intensity-weighted particle size distribution. FIG. 5 and FIG. 6 show representative distributions for nanoscale hydrogels in the swollen and collapsed state, suggesting the cumulant analysis is applicable for these measurements. It should be noted that both CONTIN and NNLS are mathematically equipped to describe multimodal particle distributions.

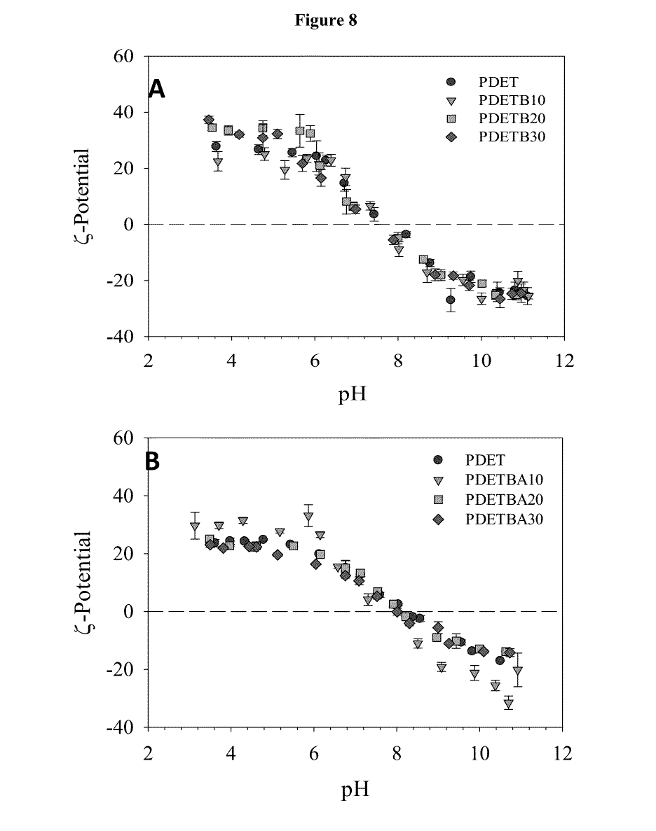

The colloidal stability of nanoparticle dispersions is a function of both surface charge and/or any steric stabilization from adsorbed or bound molecules protruding from the surface. A net surface charge, or ξ-potential, of ±30 mV is generally regarded as the minimum for purely electrostatic stabilization. The resistance to particle-particle aggregation was tested in a copolymer containing a hydrophobic co-monomer, TBMA, after 4 weeks and 8 weeks in aqueous suspension. The TBMA was incorporated to increase network core hydrophobicity, and as such, these nanoparticles should display the highest propensity for aggregation or flocculation. According to DLVO theory, this process in colloidal suspensions is driven largely by Van der Waals attractions between approaching particles undergoing Brownian motion. The results, shown in FIG. 8, demonstrate that the nanogels are able to resist aggregation for at least 8 weeks in aqueous suspension. The hydrodynamic diameter remains at approximately 93-96 nm in the deswollen state (pH>7.5) and 119-122 nm in the swollen state (pH<6.0). The pH_{c} is also unchanged from 4 to 8 weeks in aqueous suspension.

The polydispersity index (PdI), is given by a ratio of the second (μ₂) and first moment (F) of the Cumulants analysis (μ₂/Γ²) and describes the apparent width of the size distribution. It should be noted that this PdI, as defined in the Cumulants analysis, does not describe a true particle size distribution, but rather the width of an assumed Gaussian distribution around a single exponential fit of the generated autocorrelation function. Little variation is seen in the PdI between 4 weeks and 8 weeks in aqueous suspension. Moreover, nearly all PdI values, except measurements in the hydrophobic to hydrophilic phase transition around pH 7.0, lie below 0.2. These data suggest our nanogels are stable in aqueous suspension and are able to resist significant particle aggregation over the course of at least 8 weeks. When we replaced reactive PEGMA (MW˜2080) with a non-reactive PEG monomethyl ether (MW˜1900) in the photoemulsion reaction mixture, the resultant nanogels (w/o PEG graft) demonstrated poor stability and underwent visible flocculation and sedimentation within 1 h. As a result, we infer that the PEG graft is important to conferring colloidal stability to these nanogels.

Evaluation of pH-Responsive Phase Transition

Dynamic Light Scattering.

Dynamic light scattering was used to probe the volume swelling transition of the nanogels, including their swelling ratio and critical swelling pH. The latter is of particular interest in hydrogel-mediated intracellular drug delivery because this parameter is an indication of physiological pH (endosomal vs. extracellular) at which the network swells and permits drug efflux to the surrounding milieu.

In the pH-dependent phase transitions described in the following text, nanogels were first adjusted to pH 10.5 and titrated to progressively lower pH values using HCl. The justification is that acidification (via the action of endosomal ATP-dependent proton pumps) is the most prevalent pH-dependent pathway an intracellular drug delivery carrier would experience. In our tests, the pH-dependent swelling response was completely reversible with no evidence of hysteresis upon titrating from pH 10.5 to pH 3.5 and back to pH 10.5. No salt induced flocculation was observed in these studies over the course of two titration cycles.

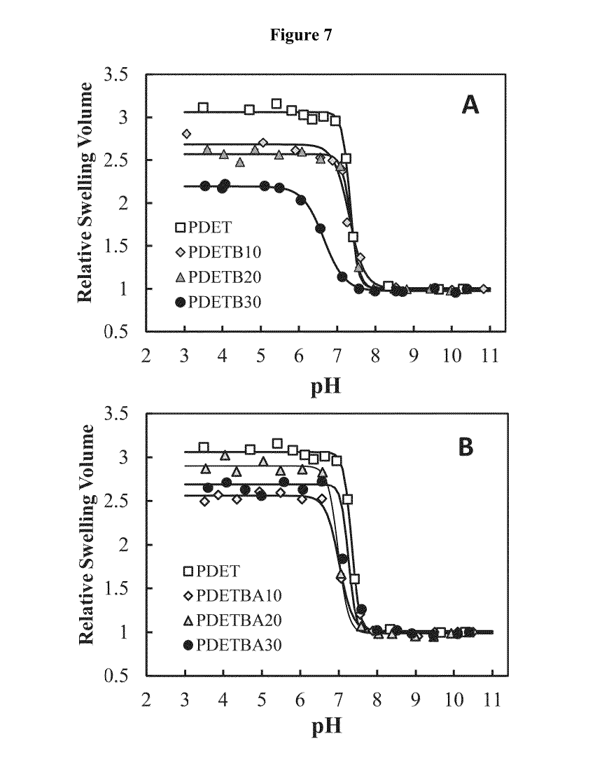

FIG. 7 illustrates the influence of hydrophobic moiety incorporation on pH-dependent volume swelling of the nanogel formulations. Swelling in ionizable hydrogel systems is driven by a balance of thermodynamic and physical forces; namely the free energy of polymer and solvent interactions, osmotic pressure generated by mobile counterions inside the gel, and elastic contractile response to gel deformation. As hydrophobic content increases, greater proton activity or greater ionization (i.e. lower pH) is required to promote polymer/solvent/ion interaction over polymer/polymer interaction. As expected, this effect also leads to a decrease in the onset of pH-dependent gel swelling.

The addition of TBMA (FIG. 7, Panel A) clearly shifts the onset of pH-dependent swelling from ˜pH 7.8 to pH 7.0. Moreover, the critical swelling pH can be defined by fitting a hyperbolic tangent or sigmoidal function to the measured hydrodynamic diameter (D_{H}) and determining the inflection point. In this case, data were fit to a hyperbolic tangent function of the form

D_{H}=A+B·tan h(C·pH+D) (1)

Taking the second derivative of Equation 1 yields the inflection point of the curve, which is taken to represent the critical swelling pH, pH_{c}.

$${pH}_{c} = - \frac{D}{C} \qquad ( 2 )$$

In networks containing TBMA, the pH_{c} trend decreased as follows: PDET (pH_{c}=7.37)˜PDETB20 (pH_{c}=7.36)˜PDETB10 (pH_{c}=7.31)>PDETB30 (pH_{c}=6.65). Additionally, PDET, PDET10, and PDET20 have reached maximum swelling volume by ˜pH 6.8, while PDETB30 reaches maximum volume swelling near pH 6.0. This pH is characteristic of the early endosomes in mammalian cells, illustrating that these networks may have utility as vehicles for intracellular drug delivery.

The degree of volume swelling is decreased as the gel concentration of TBMA is increased. Both PDETB10 and PDETB20 exhibit a lower volume swelling ratio than the base formulation PDET, while PDETB30 exhibits a markedly reduced capacity for network expansion. This observation may be ascribed to two effects, (1) the persistence of hydrophobic associations in the polymer network that resist solvation and limit elastic deformation of the network, and (2) a reduction in ionizable amine content and consequent decrease in osmotic pressure generated by salts migrating into the network core.

In order to maintain the total monomer concentration constant throughout various formulations, an increase in TBMA concentration required a concomitant decrease in DEAEMA concentration. Simply, the PDETB30 contains few tertiary amines than PDET, and therefore, the capacity for electrostatic repulsion and osmotic swelling decreases proportionally with decreasing DEAEMA content. These data suggest that there may be a critical [TBMA]:[DEAEMA] ratio, below which hydrophobic TBMA groups are not present in sufficient concentration to segregate into hydrophobic domains and counterbalance the effects of amine-group ionization and osmotic swelling.

Incorporation of TBAEMA (FIG. 7, Panel B) also lowers the onset of pH-dependent swelling, though the effect is less pronounced. In these copolymers, the decrease in pH_{A} does not follow any particular compositional trend: PDET (pH_{c}=7.37)>PDETBA30 (pH_{c}=7.13)>PDETBA10 (pH_{c}=7.01)˜PDETBA20 (pH_{c}=6.98). These formulations also display similar capacity for volumetric swelling. As TBAEMA contains a secondary amine, the network charge density (and resultant osmotic pressure) should remain relatively constant between PDET, PDETBA10, PDETBA20 and PDETBA30. Moreover, this suggests the ionizable secondary amine group in TBAEMA likely precludes the formation of discrete domains of hydrophobic t-butyl groups. Hydrophobic domains in polyelectrolyte gels have been shown to increase the effective crosslinking density and reduce volumetric swelling. These data suggest that the presence of a t-butyl-R group is not necessary and sufficient to depress pH_{c}, but rather a more general, hydrophobic effect governs the modification of phase change behavior.

Electrophoretic Light Scattering.

Measurements of the effective surface ξ-potential reveal insignificant differences between the various formulations. These data are consistent with the expectation that the PEG-grafted surface of these nanoscale hydrogels are very similar and that the modifications in monomer composition primarily affect the network core. All formulations possess a reversible surface charge, with an isoelectric point (IEP) at approximately pH 8.0, slightly positive ξ-potential at pH 7.4, and a maximum ξ-potential of 25-30 mV at pH 3.50 (FIG. 8).

At physiological pH, the slightly positive ξ-potential may help facilitate non-specific cell-uptake. The negative ξ-potential observed from pH 10.5 to ˜pH 8.0 can be ascribed to the adsorption of negatively charged hydroxyl ions on the PEG-coated surface and has been noted previously in similar DEAEMA-based materials. Likewise, the positive ξ-potential can be ascribed the surface adsorption of hydronium ions and protonation of amine-containing groups in the network core, which serve to establish an electrical double layer around the particles. While the measured ξ-potential for all formulations fall outside the limits for electrostatically-driven colloidal stability (±30 mV), no flocculation or aggregation was observed throughout the pH range, even that where nanoscale hydrogels possessed a net surface charge of ±5 mV. This provides additional evidence of the steric stabilization afforded by PEG surface grafts. Moreover, previous work has estimated the ξ-potential of neat PDEAEMA nanogels to be approximately −45 mV at pH 10 and 75 mV at pH 4. Thus, the surface layer of PEG was indeed effective at shielding surface charge.

Pyrene Fluorescence Spectroscopy.

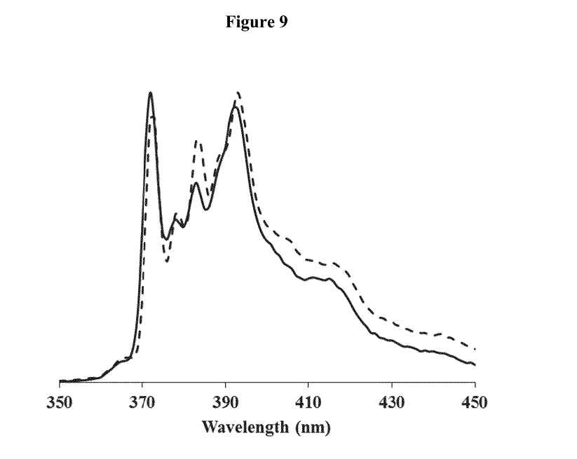

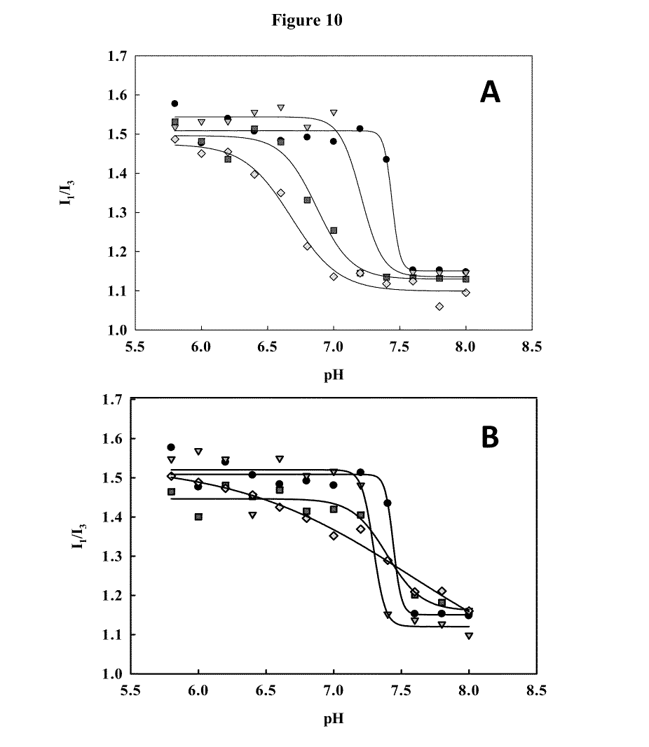

The ratio of the first to third vibronic peak (I₁/I₃) in the fluorescence emission spectra of pyrene was used to study the pH-dependent conformational transition of responsive nanoscale hydrogels. The fluorescence spectra of pyrene undergo a characteristic shift depending on the polarity of pyrene microenvironment. When dissolved in highly polar, aqueous solvents the I₁/I₃ ratio in the emission spectra is approximately 1.59 while this ratio decreases to 0.61 in nonpolar, aliphatic hydrocarbons such as n-hexane or dodecane. Therefore, an increase in the emission I₁/I₃ ratio indicates pyrene is preferentially partitioned in hydrophobic domains. FIG. 9 shows a representative change in pyrene fluorescence in aqueous suspensions of PDETB30 between collapsed hydrophobe (pH 8.0) and swollen hydrophile (pH 6.0). Consequently, the fluorescence spectra of pyrene can be used to probe the polarity of aqueous suspensions of nanoscale hydrogels and determine the influence of polymer composition on relative network hydrophobicity and the critical pH required to induce a conformational transition. FIG. 10A clearly demonstrates that inclusion of TBMA causes, in a composition-dependent fashion, a decrease in the pH required to induce a conformational transition.

This value decreases from approximately pH 7.5 in PDET to below pH 7.0 in PDETB30, with both PDETB10 and PDETB20 exhibiting intermediate values. The measured pKa values for PDET and PDETB30 are 7.3 and 6.9, respectively. The pKa for PDET is in excellent agreement with previous reports of DEAEMA-based polymers. The decrease in polymer pKa is expected as increased network hydrophobicity is known to lower the pKa of ionizable amines.

Additionally, whereas PDET displays a rather abrupt transition from hydrophobe to hydrophile, the networks containing TBMA transition over a substantially wider pH range. In bulk gels of similar monomeric composition P(DMAEMA-co-TBMA) to our nanogels P(DEAEMA-co-TBMA-g-PEGMA), the persistence of hydrophobic microdomains resulted in 35% increase in shear modulus at 20% TBMA content and a 245% increase at 40% TBMA content. In the present study, a similar stiffening effect could be imparted by TBMA in DEAEMA-based nanogels and result in a more broad swelling transition.

These studies also provide additional insight into the relative hydrophobicity of the network core. For example, at pH 8.0, the I₁/I₃ ratio for collapsed PDET is 1.15 whereas the I₁/I₃ ratio for collapsed PDETB30 is approximately 1.09. Upon suspension in pH 5.8 buffers, these ratios increased to 1.58 and 1.49 for PDET and PDETB30, respectively. These values for I₁/I₃ correspond well with previous reports of pyrene fluorescent probes in DEAEMA-based polymeric materials.

Our understanding of the impact of TBAEMA on pH-dependent swelling transition is less clear, however. Nanogels containing TBAEMA did not exhibit the same type of composition-dependent phase transition displayed by the TBMA copolymers (FIG. 10B). When copolymerized in DEAEMA-based nanogels at low concentrations (PDETBA10), TBAEMA decreases the critical swelling pH in both light scattering (FIG. 7B) and pyrene (FIG. 10B) studies. The breadth of the transition remains relatively constant when compared to PDET, which contains neither TBAEMA nor TBMA. When copolymerized with DEAEMA at 20 mol % and 30 mol % (PDETBA20 and PDETBA30), TBAEMEA raises the pH, as determined by DLS and significantly increases the breadth of the hydrophobe-hydrophile phase transition as determined by pyrene fluorescence. This observation is seemingly inconsistent with the chain stiffness argument applied to TBMA-containing nanogels. If chain stiffness and mobility were the dominant factors governing the breadth of the hydrophobe-hydrophile phase transition, one would expect PDET, PDETBA10, PDETBA20, and PDETBA30 to have phase transitions of similar breadth. DEAEMA and TBAEMA have identical molecular weights and similar end-group bulkiness. We therefore expect that TBAEMA will have little impact on the chain stiffness.

One possible explanation for the broad nature of the TBAEMA-induced hydrophobe-hydrophile phase transition may be the heterogeneous distribution of ionizable amine species in the network core. Titration studies reveal two buffering regions for PDETBA30; the midpoints occur at pH 7.3 and pH 8.3. These values agree with previous reports for PDEAEMA with pKa˜7.0-7.3 and PTBAEMA with pKa˜7.6-8.0. Thus, PDETBA30 should possess a greater network charge density at elevated pH and create an increasingly polar environment (indicated by increasing I₁/I₃ in FIG. 10B) that is distributed over the pKa range of the multiple ionizable species. This effect is less notable in PDETAB20 and nearly absent in PDETBA10. Comparative hydrophobic to hydrophilic phase transitions between PDET, PDETB30, and PDETBA30 are shown in FIG. 11.

Conclusions.

Nanoscale, pH-responsive polycationic networks were successfully synthesized using a photoemulsion polymerization. Copolymer composition and incorporation of hydrophobic moieties, TBMA and TBAEMA, was verified using ¹H-NMR. Hydrogel nanoparticles exhibit a dry diameter of approximately 50-65 nm as determined by TEM and a collapsed, yet hydrated, diameter of approximately 90 nm. Dynamic light scattering reveals a single distribution of particle sizes that remain stable in aqueous suspension for at least 8 weeks. In P(DEAEMA-g-PEGMA) copolymers, the onset of pH-dependent swelling occurs ˜pH 7.8-8.0 and networks have reached maximum volume swelling ˜pH 6.7-7.0. In P(DEAEMA-co-TBMA-g-PEGMA) copolymers, the onset of pH-dependent swelling decreasing with increasing TBMA content, reaching ˜pH 7.2 with maximum volume swelling ˜pH 5.50 in PDETB30. Moreover, TBMA broadens the transition from collapsed hydrophobe and swollen hydrophile in light scattering and pyrene fluorescence spectroscopy studies. In P(DEAEMA-co-TBAEMA-g-PEGMA) copolymers, the compositional dependence is less obvious and may be complicated by the presence of multiple ionizable species in TBAEMA and DEAEMA. The polymerization described in this report and in our previous work permits the reliable formation of hydrogel nanoparticles with utility in diverse applications that require tailoring material properties. In particular, PDETB30 possesses size (d_{H}˜100 nm) responsive characteristics (pH_{c}˜6.6) well-suited for intracellular drug delivery applications.

### Example 2

### Membrane Disruptive Properties of Hydrophobic Polybasic Nanoscale Hydrogels

Compositional considerations, such as the balance between cationic and nonionic, hydrophilic components and ratio of hydrophobic monomers have significant impact on resultant drug delivery properties (i.e. transfection efficiency, complex stability, etc.). It is generally understood that increasing cationic content leads to increased nucleic acid condensation. However, excess cationic content in polymer delivery systems can have deleterious effects. High cationic charge density is frequently correlated with toxicity of conventional cationic polymers like poly(ethyleneimine) (PEI) and may host of undesirable consequences in vivo.

However, excessive core hydrophobicity could result in limited siRNA encapsulation, poor buffering capacity, and inefficient endosomolysis due to lack of cationic core groups. Decreased swelling observed with increased core hydrophobicity may also limit the diffusion of siRNA in to and out of the nanogel below critical swelling pH.

This example investigates the role of hydrophobicity in modulating membrane destabilization. To study this interaction, three model membrane systems were used. Sheep erythrocytes were used to assess the pH- and concentration-dependent membrane destabilization of lipid bilayers, lactate dehydrogenase (LDH) leakage was measured from Caco-2 and RAW 264.7 cells to evaluate the non-specific membrane destabilization in live cells, and giant unilammelar vesicles (GUVs) were used to gain insight into the mechanism of membrane destabilization. Cytotoxicity of each copolymer was also investigated as a function of concentration and exposure time.

Materials and Methods

Cell Culture.

Human colorectal adenocarcinoma cells (Caco-2) and murine macrophages (RAW 264.7) were maintained in Dulbecco's Modified Eagles Medium (DMEM) supplemented with 100 U mL⁻¹ penicillin, 100 μg mL⁻¹ streptomycin, 0.25 μg mL⁻¹ Amphotercin B, and 10% FBS. Caco-2 cells were used between passage 34 and 62. RAW 264.7 cells were used between passage 9 and 16. Caco-2 cells were passaged by washing with pre-warmed Dulbecco's phosphate buffered saline (DPBS) and subsequent incubation with 0.25% Trypsin-EDTA at 37° C. Trypsin was neutralized by addition of fresh, prewarmed DMEM and cells were separated by centrifugation. The resulting pellet was suspended in 10 mL DMEM and cell count was determined using a Scepter Automated Cell Counter (Millipore, Billerica, Mass.) with 60 μm tips. The cell suspension was diluted as necessary and added to tissue-culture treated flasks or multi-well plates. Caco-2 cells were typically passaged at 1:5 ratio with media replenished every 2-3 days. RAW 264.7 cells were passaged by washing with prewarmed DPBS and replacing the original culture volume with fresh DMEM. Cells were removed from the flask surface by gentle scraping with a 25 cm cell scraper (BD Falcon, Franklin Lakes, N.J.). The number of suspended cells was counted using a Scepter Automated Cell Counter and diluted as necessary for addition to tissue culture flasks or multi-well plates. RAW 264.7 cells were typically passaged every 2 days.

Cytocompatibility.

In vitro cytocompatibility of polycationic nanoscale hydrogels was evaluated using commercially available cytotoxicity assays. MTS assays were performed using the CellTiter 96 AQueous One Solution Cell Proliferation Assay kit (Promega Corp., Madison, Wis.) in which the soluble tetrazolium salt [3-[4,5-dimethylthiazol-2-yl]-5-(3-carboxymethoxyphenyl)-2-(4-sulfophenyl)-2H-tetrazolium] (MTS) is reduced to a purple formazan product. The absorbance of the formazan product is proportional to the number of viable cells. Stock solutions of polymer were suspended in PBS and allowed to equilibrate overnight. Caco-2 cells were seeded in 96-well plates at 15,000 cells/well and incubated for 36 hours prior in 200 μL, DMEM. RAW 264.7 cells were seeded in 96-well plates at 10,000 cells/well and incubated for 36 hours prior to assay in 200 μL, DMEM. Media was aspirated and cells were washed 2× with DPBS and incubated in 160 μL A serum-free DMEM for 90 minutes. Following this incubation period, polymer stock solutions at 5× were added to cells for another designated exposure times. Media and polymer were aspirated and replaced with a DMEM/MTS solution. Absorbance at 490 nm was recorded after 4 hours incubation in the DMEM/MTS solution.

Hemolysis.

Sheep blood in sodium citrate was obtained from Hemostat Laboratories (Dixon, Calif.) and used for up to two weeks after receipt. Phosphate buffers (0.15 M) from pH 5.0-8.0 were prepared by dissolving predetermined amounts of monosodium phosphate and disodium phosphate in ultrapure DI water. The buffer pH was adjusted as needed using 1 N HCl or 1 N NaOH. Dry nanoscale hydrogels were suspended in 150 mM phosphate buffer at the desired pH at a concentration of 2.5 mg ml⁻¹ and allowed to equilibrate overnight. Erythrocytes were isolated from whole sheep blood by 3 successive washes with freshly prepared 150 mM NaCl. Red blood cells (RBCs) were separated by centrifugation from 10 minutes at 2,000×g. The supernatant and remaining buffy coat were carefully aspirated and discarded. After removing the supernatant following the final wash, RBCs were suspended in a volume of 150 mM phosphate buffer identical to that of the original blood aliquot at the pH matching that of the suspended polymers. This solution was diluted 10-fold in 150 mM phosphate buffer to yield an RBC suspension of approximately 5×10⁸ cells/mL. In a typical experiment, 1×10⁸ RBCs were exposed to nanogels at specified concentrations while shaking in a bead bath (LabArmor, Cornelius, Oreg.) pre-equilibrated at 37° C. Following a 60 min incubation period, samples were centrifuged at 14,500 RPM for 5 min to separate cells and membrane fragments. An aliquot of each sample was transferred to a clear 96-well plate and hemoglobin absorbance was measured at 541 nm. Negative controls (0% lysis) consisted of 150 mM phosphate buffer at experimental pH and positive controls (100% lysis) consisted of RBCs incubated in ultrapure DI water.

The pH values tested in this analysis range from pH 5.0-pH 8.0; experiments performed at pH 5.00, 5.50, 6.00, 6.50, 7.40, 7.60, 7.80, and 8.00. The concentrations tested range from 1-2000 μg ml⁻¹; with experiments performed with 2000, 1000, 500, 250, 100, 50, 25, 10, 5, 2.5, and 1 μg ml⁻¹ nanogel suspended in 150 mM phosphate buffer at the specified pH.

Lactate Dehydrogenase Release.

LDH assays were performed using a CytoTox-ONE™ Homogeneous Membrane Integrity Assay (Promega Corp., Madison, Wis.) to measure release of lactate dehydrogenase (LDH) from cells with damaged membranes. Cells were seeded to 96-well plates and polymer solutions added as previously described. At designated time points, 50 μL aliquots of media was aspirated and combined with 50 μL LDH assay buffer in a black-walled 96-well plate. Following 10 minutes incubation at room temperature, the fluorescence was measured at 530 ex/590 em. Generally, cell culture plates were used for a maximum of two different aliquots.

Giant Unilammelar Vesicle Disruption.

1-palmitoyl-2-oleoyl-sn-glycero-3-phosphocholine (POPC), 1,2-dihexadecanoyl-sn-glycero-3-phosphoethanolamine (DHPE) labeled with BODIPY® FL, cholesterol, and Texas Red-sucrose were kindly donated by Prof. Jeanne Stachowiak (University of Texas at Austin, Austin, Tex.). Giant unilammelar vesicles (GUVs) were synthesized via electroformation as previously described [12, 13]. Briefly, lipid/cholesterol solutions were combined in the following ratio: 7:3:0.01 POPC:Cholesterol:Bodipy FL DHPE and drop-cast onto clean glass slides. The lipid solutions were allowed to dry and were then assembled into electroformation chambers. Vesicles were electroformed at 60° C. in Texas Red-sucrose (˜350 milliosmole(mOsm)) solution.

GUVs were placed in 35 mm glass-bottom petri dishes for real-time confocal microscopy imaging. PDET and PDETB30 were prepared at 2 mg ml⁻¹ in 100 mM phosphate buffer adjusted to pH 6.50. The osmolarity of the resulting suspensions was measured and adjusted with sucrose to ˜350 mOsm as needed. 1 ml of GUV suspension was transferred to the glass-bottom petri dish and was allowed to sediment for 5 min. 25 μL of the nanogel suspension was carefully injected into the dish so as not to disturb the spatial distribution of focused GUVs. Images were collected every 5 s at a fixed focal plane.

Results and Discussion

Pyrene Fluorescence Spectroscopy.

Analogous to the pyrene emission studies performed above, pyrene excitation spectra were collected to confirm the pH required for phase transition from hydrophobe to hydrophile. The maximum excitation wavelength of pyrene red-shifts from 333 nm to 338 nm upon transition from polar to nonpolar environment. Therefore, an increase in the excitation I₃₃₈/I₃₃₃ ratio indicates pyrene is preferentially partitioned in hydrophobic domains. FIG. 12 shows a representative change in pyrene fluorescence in aqueous suspensions of PDETB30 between collapsed hydrophobe (pH 8.0) and swollen hydrophile (pH 6.0). Consequently, the fluorescence excitation spectra of pyrene can be used to probe the polarity of aqueous nanogel suspensions and determine the influence of polymer composition on nanogel hydrophobicity and the critical pH required to induce a conformational transition.

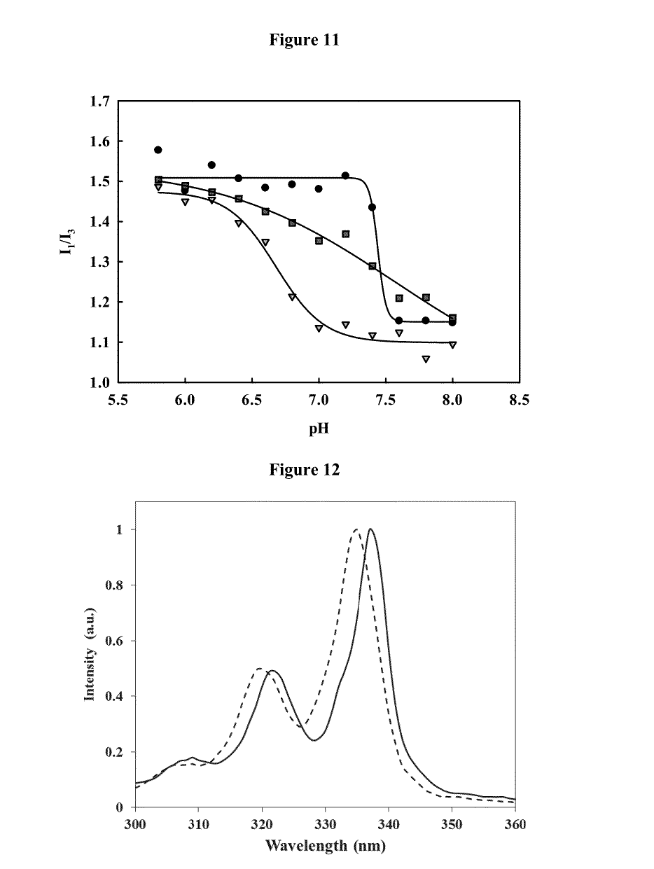

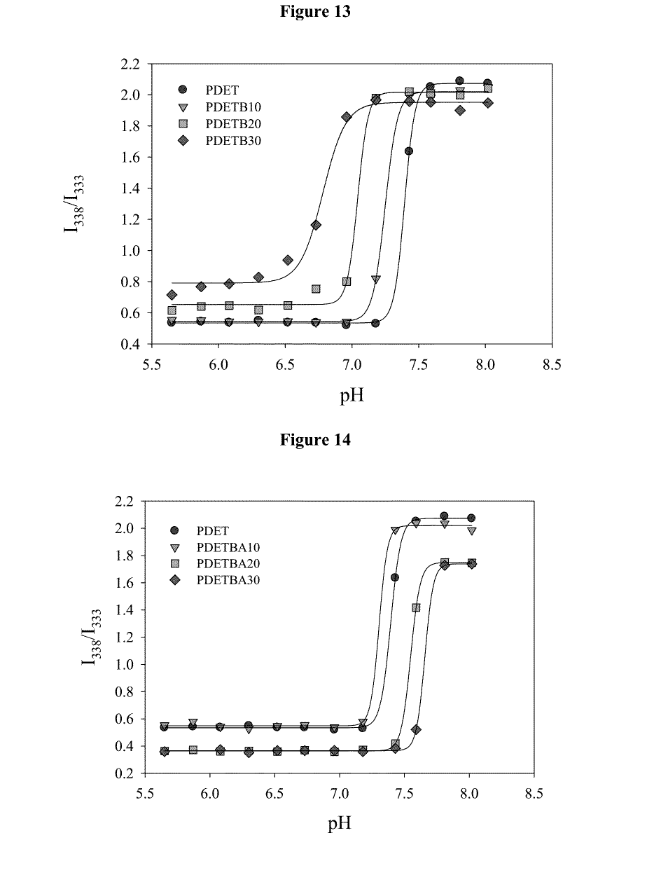

Increasing the proportion of TBMA causes a clear decrease in the onset for the pH-dependent phase transition, as seen in FIG. 13. These observations follow the expected trend—increasing TMBA composition progressively lowers the apparent pH (pHₐₚₚ) for the hydrophobic—hydrophilic transition. Moreover, these data corroborate the pH-responsive swelling profiles presented in FIG. 7A and are in excellent agreement with the pyrene emission studies shown in FIG. 10A.

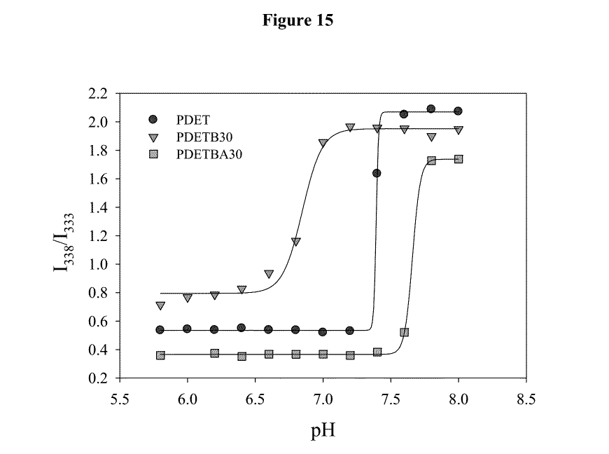

Likewise, increasing the proportion of TBAEMA from 0 mol % of DEAEMA to 30 mol % of DEAEMA causes a clear increase in the onset of the pH-dependent phase transition, as seen in FIG. 14. This trend is also expected, given that TBAEMA contains a secondary amine that should increase the pKa, and thus the onset of pH-dependent phase transition, to higher pH values. The broad transitions observed for PDETBA20 and PDETBA30 for pyrene emission studies (FIG. 10B) are notably absent in the I₃₃₈/I₃₃₃ ratio of pyrene excitation spectra. In fact, the breadth of the phase transition is quite similar for PDET, PDETBA10, PDETBA20, and PDETBA30. A direct comparison of the effects of TBMA and TBAEMA on pH of hydrophobe-hydrophile phase shift can be seen in FIG. 15.

Membrane Destabilization.

This series of experiments was constructed to identify nanogels capable of selective membrane destabilization. An optimal nanogel would be relatively inert and non-disruptive under normal physiological conditions. Upon transition to endosomal conditions, this optimal nanogel would undergo a conformational transition to render it capable of potent membrane destabilization. Conversely, a non-optimal nanogel would mediate membrane disruption under physiological conditions and/or be non-disruptive in endosomal conditions.

Hemolysis.

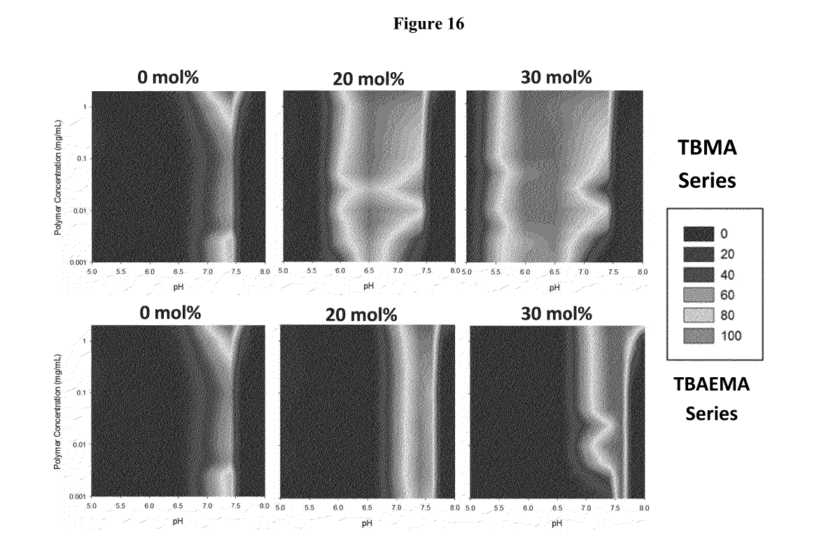

Hemolysis experiments were used to approximate the endosomolytic ability of these nanogels. The pH- and concentration-dependent hemolysis was determined according to Equation 3:

$$\% Hemolysis = \frac{{A}_{sample} - {A}_{blank}}{{A}_{max} - {A}_{blank}} \qquad ( 3 )$$

Where Aₛₐₘₚₗₑ represents RBCs exposed to polymer at a given pH and concentration, A_{blank} is the absorbance of the supernatant after RBC exposure to phosphate buffer at a given pH, and Aₘₐₓ represents maximum lysis following RBC exposure to DI water. The relative lysis for nanoscale hydrogels containing varying amounts of TBMA or TBAEMA is shown in contour plot form in FIG. 16. These data demonstrate that polymer composition has a clear impact on membrane-disruptive capabilities. As demonstrated previously with dynamic light scattering studies (FIG. 7), the simple presence of a t-butyl group alone in the copolymer is not the critical parameter for exerting control over resultant physicochemical properties. Rather, the increased network hydrophobicity of TBMA-containing nanogels seems to govern the interactions with biological membranes.

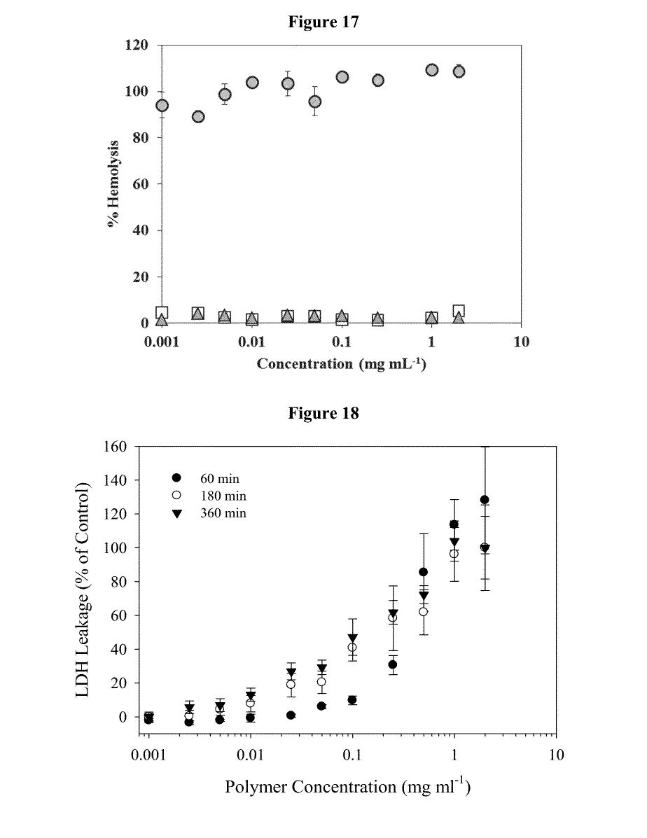

As seen in FIG. 16, inclusion of TMBA in the nanogels markedly expands both the pH and concentration range at which these networks effectively disrupt erythrocyte membranes. For example, PDET demonstrates efficient hemolysis at high concentrations (>0.25 mg mL-1) and between pH 7.0 and pH 7.6. In contrast, PDETB30 demonstrates highly efficient hemolysis in the pH range of early endosomes (pH 5.5-pH 6.5) at concentrations as low as 1 μg mL⁻¹. The enhanced hemolytic ability of PDETB30 at pH 6.0 is depicted in FIG. 17, along with that of PDET and PDETBA30. Notably, PDETB30 is 10× more efficient (on a mass basis) than previously reported polycationic block copolymer systems with demonstrated efficacy in in vitro siRNA delivery and 25× more efficient than phenylalanine-grafted pseudo-peptides with demonstrated utility in intracellular protein delivery. These data indicate that the membrane-disruptive properties of these nanogels can be tuned by adjusting hydrophobic monomer incorporation.

Lactate Dehydrogenase Leakage.

The influence of polymer composition and exposure time on membrane destabilization in live cells was investigated using an LDH membrane integrity assay. In this assay, the percentage of LDH leakage from permeabilized or damaged cell membranes can be given by an equation analogous to Equation 4.

$$\% L D H Release = 100 * \frac{{RFU}_{s} - {RFU}_{PBS}}{R F {U}_{max} - R F {U}_{P B S}} \qquad ( 4 )$$

Where RFU_{S} is the fluorescent reading from the sample, RFU_{PBS} is the fluorescent reading from cells exposed only to PBS (0% lysis) and RFUₘₐₓ (100% lysis) is the maximum fluorescent reading from the plate. In typical applications, RFUₘₐₓ is given by a commercial lysis buffer. In practice, however, the fluorescent reading generated by the greatest polymer concentration (2 mg/mL) generated fluorescent values that exceeded that of the kit lysis buffer and 1% w/v solutions of Triton-X100. Thus, LDH release is occasionally reported as >100% at polymer concentrations 1-2 mg ml⁻¹.

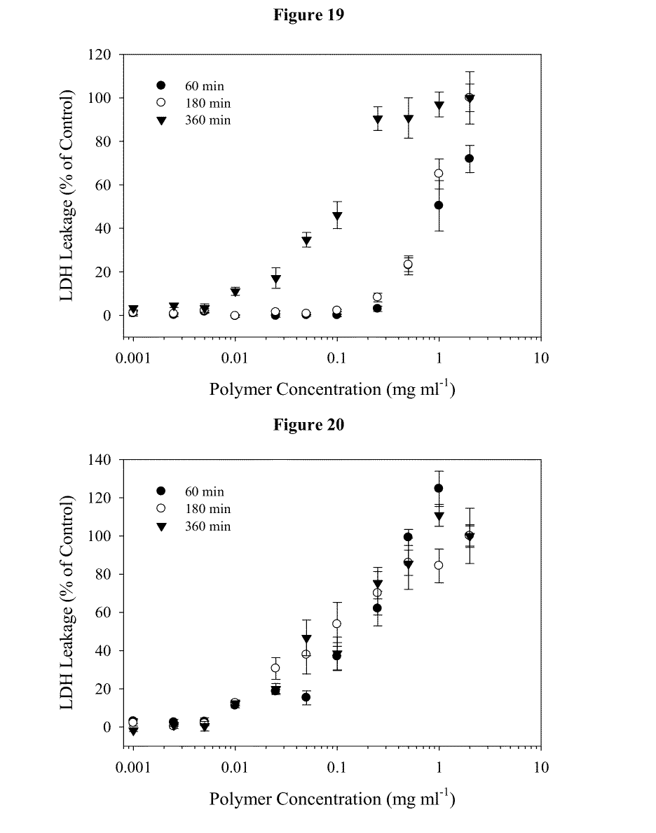

LDH leakage as a function of nanogel concentration and exposure time is shown for PDET (FIG. 18), PDETB30 (FIG. 19), and PDETBA30 (FIG. 20). For PDET (FIG. 18), the LDH leakage increases with longer exposure time (60 min to 180 min) and remains relatively constant from 180 min to 360 min. For PDETB30 (FIG. 19), the LDH leakage is negligible at concentrations up to 250 μg ml⁻¹ for 60 min and 180 min exposure. However, the leakage increases considerably following 360 min exposure. LDH release following exposure to PDETBA30 (FIG. 20) follows no clear time dependence and the release values are similar across all time points. These data underscore the need for careful consideration of incubation time in future cytotoxicity and siRNA delivery experiments to minimize the non-selective disruption of cellular membranes.

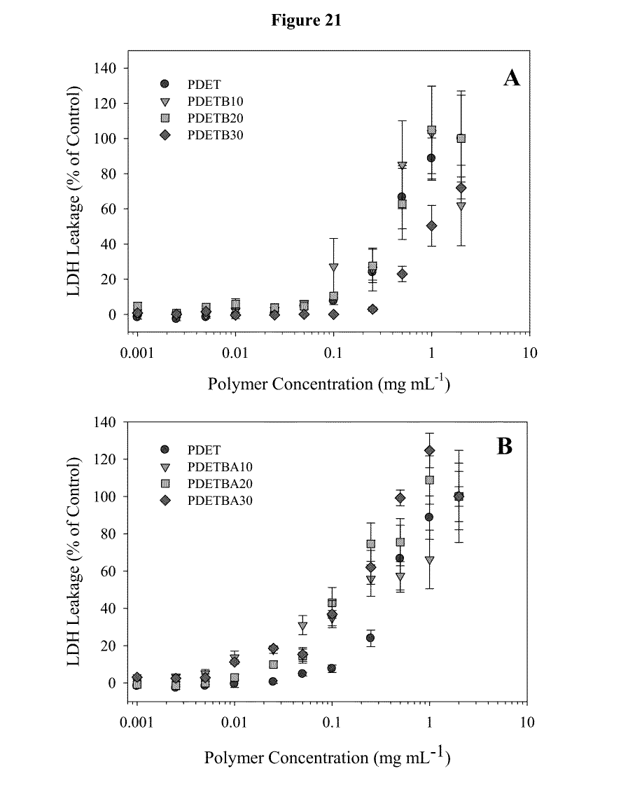

The influence of nanogel composition on LDH leakage, shown in FIG. 21A for TBMA-containing polymers and FIG. 21B for TBAEMA-containing polymers, show that PDETB30 is less damaging to Caco-2 cell membranes than PDET, PDETB10, and PDETB20. The general trend for inducing LDH membrane leakage is PDET˜PDETB10˜PDETB20>PDETB30. For the TBAEMA-containing polymers (FIG. 21B), the general trend is as follows: PDETBA30>PDETBA20˜PDETBA10>PDET. Notably, these trends are in excellent agreement with the trends in hydrophobic-hydrophilic phase transition shown in FIG. 13 and FIG. 14 and summarized in Table 6.

Therefore, the pH-responsive transition regime (from collapsed hydrophobe to swollen hydrophile) is critical factor in determining the membrane-disruptive ability of these nanogels. In all cases, nanogels were demonstrated maximum hemolysis at or near the pHₐₚₚ determined by pyrene fluorescence studies (Table 6). If this pHₐₚₚ is near physiological pH, this membrane-disruptive effect was obvious in hemolysis (at pH 7.4) and LDH leakage assays. However, if the pHₐₚₚ is decreased through increased polymer hydrophobicity (e.g. PDETB30), the nanogels are less disruptive at physiological conditions and more disruptive at endosomal conditions.

TABLE 6
Comparison of critical pH values for phase
transition and pH of maximum hemolysis.

| | | mol | | | |
|---|---|---|---|---|---|
| | | t-butyl/ | | | |
| | | 100 mol | | | |
| Name | Copolymer | DEAEMA | pH_{c}¹ | pHₐₚₚ¹ | pHₕₑₘₒ³ |
| PDET | P(DEAEMA- | 0 | 7.37 | 7.39 | 7.4 |
| | g-PEGMA) | | | | |
| PDETB10 | P(DEAEMA- | 10 | 7.31 | 7.24 | 7.4 |
| | co-TBMA- | | | | |
| | g-PEGMA) | | | | |
| PDETB20 | P(DEAEMA- | 20 | 7.36 | 7.04 | 6.5 |
| | co-TBMA- | | | | |
| | g-PEGMA) | | | | |
| PDETB30 | P(DEAEMA- | 30 | 6.65 | 6.78 | 6.5 |
| | co-TBMA- | | | | |
| | g-PEGMA) | | | | |
| PDETBA10 | P(DEAEMA- | 10 | 7.01 | 7.30 | 7.4 |
| | co-TBAEMA- | | | | |
| | g-PEGMA) | | | | |
| PDETBA20 | P(DEAEMA- | 20 | 6.98 | 7.54 | 7.4 |
| | co-TBAEMA- | | | | |
| | g-PEGMA) | | | | |
| PDETBA30 | P(DEAEMA- | 30 | 7.13 | 7.66 | 7.6 |
| | co-TBAEMA- | | | | |
| | g-PEGMA) | | | | |

¹Critical swelling pH determined by dynamic light scattering.
²pH value for apparent hydrophobe-hydrophile phase transition (pHₐₚₚ) determined by pyrene fluorescence spectroscopy. Determined by calculating the inflection point of sigmoidal fit in FIGS. 5.2 and 5.3.
³pH for maximum hemolysis at polymer concentration of 0.05 mg mL⁻¹

Giant Unilammelar Vesicle Disruption.

Visualizing a model lipid bilayer during the destabilization can provide some insight into the mechanism of membrane disruption. Prevailing theories for membrane disruptive mechanisms by cationic polymers include reorientation of lipid head groups through ammonium-phosphate interactions, transient nanopore formation following electrostatic attraction between polycation and cell membrane, or even catastrophic membrane disruption. Naturally, size, surface charge, and ligand functionalization play important roles in modulating membrane interaction. However, many of these studies rely on biophysical measurements of controlled model systems such as supported lipid bilayers. In reality, mammalian cell membranes are far more complex than these model systems. Mammalian cell membranes typically contain dynamic combinations of surface- and transmembrane proteins, sugar coatings, diverse lipid combinations, and cholesterol. Unfortunately, a mechanistic comprehension of membrane destabilization in mammalian cell membrane is currently underdeveloped.

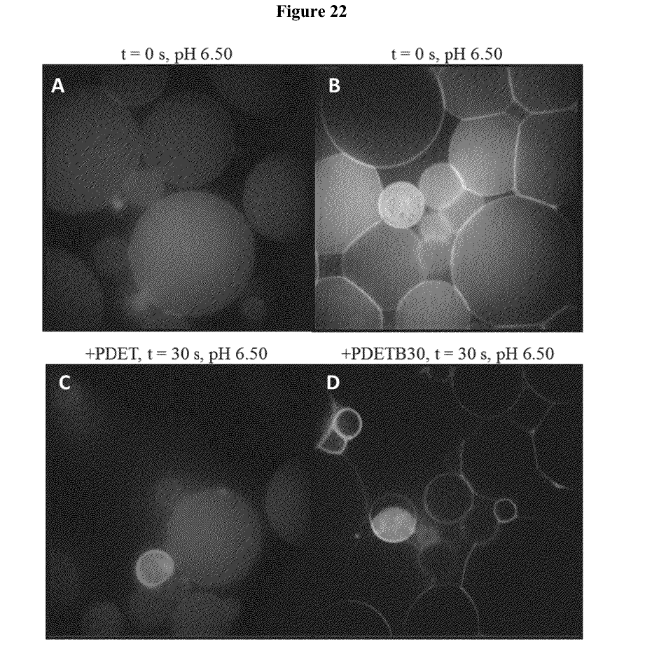

The micrographs in FIG. 22 suggest that transient nanopore formation is the predominant mechanism through which PDETB30 exerts a membrane-destabilizing effect. For these initial studies, pH 6.50 was selected to approximate the pH of an early endosomal environment. Based on the hemolysis, PDET should be non-disruptive and PDETB30 should be highly-disruptive at these conditions. Following an injection to bring the PDET to 50 μg ml⁻¹ in the buffered GUV solution, no discernible change was detected in membrane integrity. The sucrose-Texas Red remains entrapped in the GUV for several minutes after injection, confirming the persistence of membrane integrity. The difference in GUV location in FIG. 22, Panels A and C is due to a mechanical disturbance displacing the GUVs from the focal plane. Shifting the focal plane revealed that many GUVs had been forced downward by the force of the PDET injection.

In contrast, the micrographs in FIG. 22, Panels B and D, reveal substantial PDETB30-mediated destabilization of lipid membranes. Exposure to 50 ug ml⁻¹ PDETB30 in pH 6.50 buffered solution resulted in a rapid and complete efflux of sucrose-Texas Red from the vesicle interior. These data concur with the hemolysis data for PDETB30 at this concentration and pH, which indicate complete (˜100%) disruption of erythrocytes. Further efforts in this area will determine if the mechanism of membrane destabilization exhibits a dependence on polymer concentration. Additionally, the molecular weight of the entrapped fluorescent solute (currently sucrose) will be varied to estimate the average pore size formed by membrane-disruptive nanogels.

Cytocompatibility.

The effect of polymer concentration and composition on cellular proliferation was assessed using MTS assays. These data are important to determine the non-toxic polymer doses for future drug delivery experiments. In this assay, the metabolic activity of an experimental population relative to control populations can be given by the ratio:

$$Relative Proliferation = \frac{{A}_{s} - {A}_{bkg}}{{A}_{PBS} - {A}_{bkg}} \qquad ( 5 )$$

Where Aₛ is the absorbance (λ=490 nm) from sample wells, A_{bkg} is the background absorbance from DMEM/MTS solution, and A_{PBs} is the absorbance from wells in which cells were incubated only with DPBS.

Caco-2 Cells.

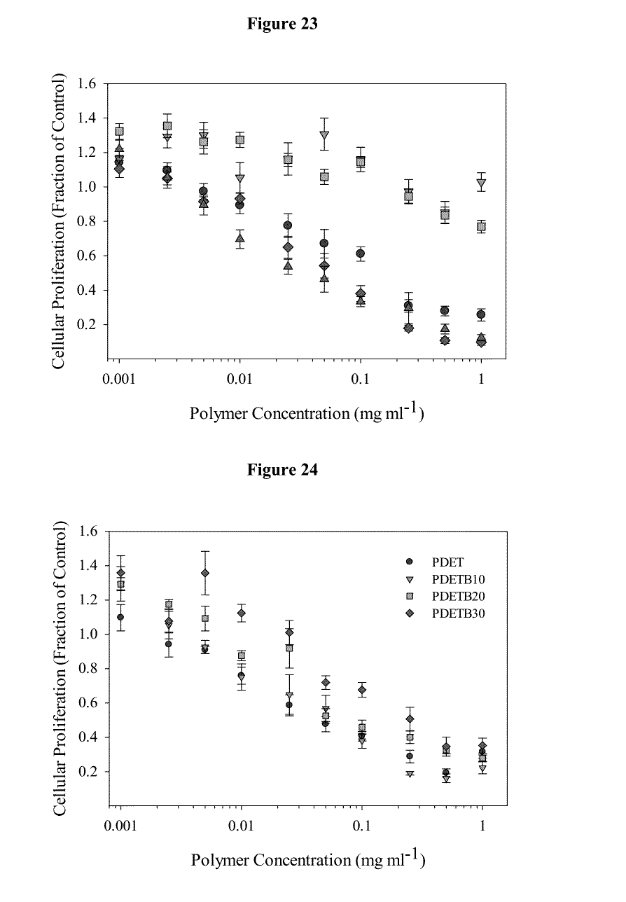

As seen in FIG. 23, PDETB20 and PDETB30 are non-toxic to Caco-2 cells at concentrations below 0.5 mg mL⁻¹. From 0.05 mg mL⁻¹-2 mg mL⁻¹, these formulations are significantly less toxic than the base formulation of P(DEAEMA-g-PEG) (PDET). It has been well documented that free amino groups contribute to the untoward cytotoxicity of many polycationic delivery agents and that increased cationic charge density correlates with increased cytotoxicity. As expected, polymers with similar cationic charge densities, e.g. nanogels with 20 mol % and 30 mol % TBAEMA, as well as PDET, exhibit similar toxicity profiles. By nature of the polymer composition, nanogels with 20 mol % and 30 mol % TBMA have less cationic charge density and thus result in decreased toxicity.

RAW 264.7 Cells.

In order to assess the concentration- and time-dependent toxicity of polymer carriers in model cells of intestinal phagocytes, MTS assays were conducted on murine macrophage cells. As seen in FIG. 24, the composition-dependent trend in toxicity profile remains consistent with observations in Caco-2 cells, though the magnitude of difference in toxicity was less pronounced. The general trend across the concentration range, in terms of relative toxicity, was PDET>PDETB10>PDETB20>PDETB30. PDETB30 was significantly less toxic (p<0.05) than the base formulation of PDET from 5-500 μg mL⁻¹.

Comparison the polymer dose-dependent toxicity between Caco-2 cells and RAW 264.7 cells reveals that the macrophages are more sensitive to the presence of nanogels than are the Caco-2 cells. This disparity may be due to the relative amount of nanogel uptake demonstrated by each cell type. Macrophages are phagocytic cells and will more readily imbibe macromolecules from their environment, thereby amplifying any harmful effects of the nanogels on cellular membranes or processes.

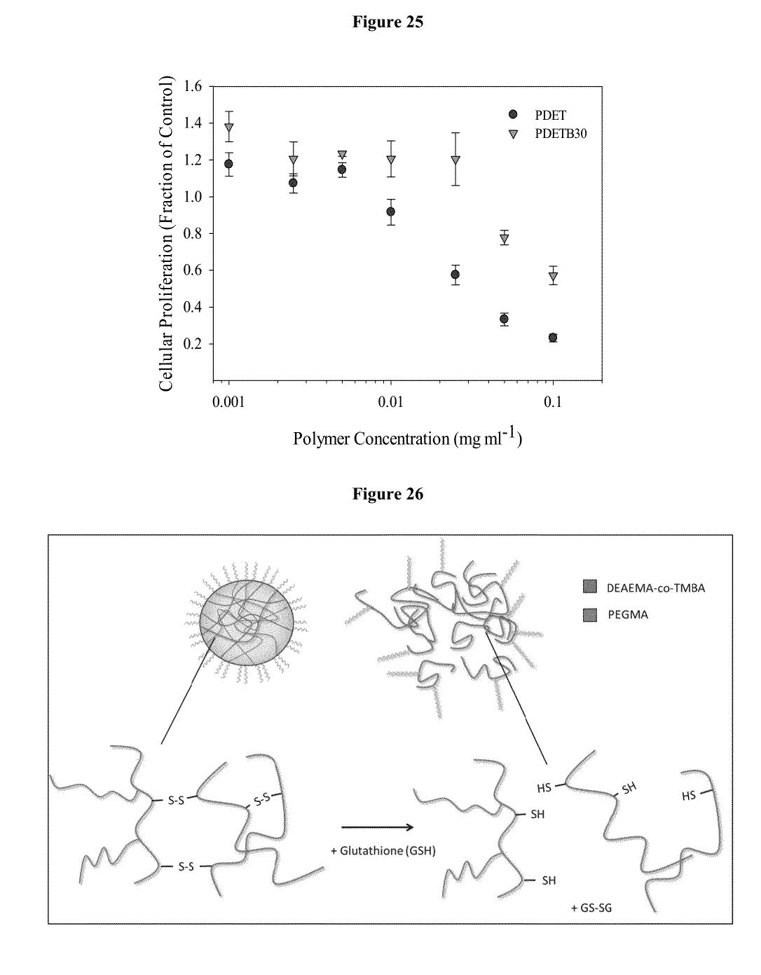

In order to develop a conservative estimate for nontoxic nanogel concentrations in further experiments, RAW 264.7 cells were exposed to nanogels for 24 h and the metabolic activity compared to untreated control cells via MTS assay. This comparison, shown in FIG. 25 for PDET and PDETB30, reveals that PDETB30 is non-toxic below nanogel concentrations of 50 μg ml⁻¹.

Conclusions

Physicochemical properties of nanoscale hydrogel networks, including critical phase transition pH, membrane disruption, and cytocompatibility can be modulated by tuning polymer composition. PDETB30 nanogels exhibit favorable pH-responsive phase transition behavior for intracellular delivery and offer an excellent combination of hemolytic ability and cytocompatibility. Additionally, the breadth of the pH range for maximum membrane disruption is related to the pH range for hydrophobic-hydrophilic transition. PDETB30 is membrane-disruptive over a broader pH range than other nanogels that undergo a more rapid hydrophobic-hydrophilic phase transition (e.g. PDET and PDETBA30). For these reasons, TBMA-containing nanoscale hydrogels, particularly PDETB30, possess attractive characteristics for intracellular drug delivery vehicles.

### Example

### Cytoplasmic Delivery of Functional siRNA Using pH-Responsive Nanoscale Hydrogels

The nanogel with the most promising attributes for siRNA delivery consists of a (1) ionizable core of 2-(diethylaminoethyl methacrylate) (DEAEMA), (2) hydrophobic comonomer of tert-butyl methacrylate (TBMA), and (3) grafted corona of poly(ethylene glycol). This nanogel (PDETB30) undergoes a volume phase transition from collapsed hydrophobe to swollen hydrophile at approximately pH 6.5 and is highly disruptive to model membrane systems in this transition region. Additionally, PDETB30 displays excellent biocompatibility to Caco-2 cell and RAW 264.7 cells in in vitro toxicity assays.

However, a critical aspect of intracellular delivery systems is a mechanism for elimination or degradation. Disulfide linkers can be cleaved by the reductive tripeptide glutathione (GSH); present at intracellular concentrations of 1-11 mM. By incorporating these linkers into polycationic nanogels, we can impart degradability to the network while retaining their mechanical integrity and pH-responsive behavior (FIG. 26).

This chapter describes the synthesis and characterization of nanogels with reducible disulfide crosslinks and compares their suitability as siRNA carriers with non-degradable PDETB30 nanogels.

Materials and Methods

Synthesis of Disulfide Crosslinker.

Dichloromethane (>99.5%) was purchased from Fisher Scientific (Plainfield, N.J.). Methacryloyl chloride (97%) and anhydrous pyridine (99.8%) were purchased from Sigma-Aldrich (St. Louis, Mo.). 2-Hydroxyethyldisulfide (90%) was purchased from Acros (Geel, Belgium).

The homobifunctional disulfide crosslinker, bis(2-methacryloyloxyethyl) disulfide (SSXL), was synthesized as follows. Organic solvents were dried over MgSO₄ before use. Dichloromethane was purged with N₂ for 15 min and placed in a dry nitrogen atmosphere (O₂<0.1 ppm, H₂O<0.1 ppm). Pyridine (15.8 mL, 0.195 mol) and bis(2-hydroxyethyl) disulfide (10.00 g, 0.065 mol) were added to cold (4° C.) dichloromethane and agitated briefly. Methacryloyl chloride was added dropwise to the stirring organic mixture over the course over 20 min. The flask was then sealed and removed from the ice bath and the reaction was allowed to proceed for 12 h in a nitrogen atmosphere.

The crude reaction product, in dichloromethane, was successively washed with 1 N HCl, 1 N NaOH, and DI water. The organic phase was retained and dried to a viscous yellow liquid via rotary evaporation. The product was then dissolved in diethyl ether and passed through a column of sodium carbonate and basic alumina. Diethyl ether was removed through rotary evaporation, again yielding a viscous yellow liquid.

Additional flash chromatography purification was performed using a Teledyne-Isco Companion Automated Flash Chromatography Instrument (Lincoln, Nebr.) equipped with a 100 g silica column. The solvent gradient was established as follows: Solvent A—hexanes, Solvent B—ethyl acetate. The gradient was adjusted from 0-15% B over 40 minutes and then from 15-100% B over 10 minutes. Fractions of interest were determined by monitoring absorbance at 258 nm. Product fractions were pooled and solvent was removed by rotary evaporation to yield a viscous, yellow liquid.

Characterization of Disulfide Crosslinker.

The composition of raw materials, purification fractions, and final product of the SSXL synthesis were investigated using a Varian (Palo Alto, Calif.) DirectDrive 400 MHz nuclear magnetic resonance spectrometer equipped with automatic sampler. Chloroform-d (CDCl₃, 99.8%) was obtained from Acros Organics (Fairlawn, N.J.). All glassware, including NMR Tubes (Wilmad Lab Glass, Vineland, N.J.), 2 mL sample vials, and Pasteur pipettes were dried overnight in a vacuum oven. SSXL was dissolved at 1% (vol/vol) in CDC₁₃ for 1H-NMR analysis. All NMR Spectra were analyzed using SpinWorks 3™ software.

Polymer Synthesis

Synthesis of Degradable Nanogel.

To impart a mechanism for biodegradation to the responsive nanogel PDETB30, SSXL was used as a replacement for tetra(ethylene glycol) dimethacrylate (TEGDMA) in the photoemulsion polymerization. SSXL was added to pre-polymer mixture at 2.5 mol % of total monomer and photoemulsion polymerization and nanogel purification proceeded as described in above.

Fluorescent Polymer Synthesis.

A fluorescent version of PDESSB30 was synthesized and purified as in identical fashion to the description in Section 6.2.1. Like the synthesis of PDETB30-OG488, the covalent conjugation of Oregon Green 488 (OG488) was enabled by the incorporation of primary amines in the PDESSB30 core. Again, 2-aminoethyl methacrylate hydrochloride (AEMA) was included in the pre-polymer feed mixture at 5 mol % of DEAEMA. The resulting copolymer was named PDESSB30f to signify the amine functionality. The primary amine of AEMA was verified with a fluorescamine assay after synthesis and purification.

Oregon Green 488 carboxylic acid, succinimdyl ester (OG488, Molecular Probes, Eugene, Oreg.) was dissolved in DMSO to yield a 10 mg ml⁻¹ solution. To form the fluorescent polymer conjugate, PDESSB30f was suspended at 10 mg ml⁻¹ in 150 mM sodium bicarbonate buffer, pH 8.30. OG488 was added to the PDESSB30f suspension to give a 1:1 mol ratio between AEMA and OG488. The reaction was stirred gently in the dark for 6 h. Following reaction completion, unreacted dye was separated from labeled PDESSB30-OG488 through dialysis against DI water. Dialysis proceeded for 3 days in 12,000-14,000 MWCO dialysis tubing (Spectrum Labs, Rancho Dominguez, Calif.) for 3 days. Labeled nanogels, PDESSB30-OG488, were lyophilized in the dark for 3 days.

Degradable Nanogel Characterization.

Several characterization techniques were employed to study the physicochemical properties of the PDESSB30 nanogels in comparison to the TMBA analogue, PDETB30. Dynamic light scattering was used to determine the hydrodynamic diameter of PDESSB30 as a function on environmental pH and was performed as described above. Measurements of the ξ-potential were performed to evaluate the effective surface charge as function of environmental pH and were performed as described above. Transmission electron microscopy (TEM) was used to determine the diameter of the dry nanogels and was conducted as described above. Evaluations of RAW 264.7 metabolic activity upon exposure to PDESSB30 and PDETB30 were conducted using a commercially-available MTS assay kits as described in above.

RNA Binding.

RNA complexation buffer was prepared by dissolving 3.15 g sodium phosphate dibasic heptahydrate, 0.02 g potassium phosphate monobasic monohydrate, 0.20 g potassium chloride, and 8.01 g sodium chloride in Milli-Q purified water. Following salt dissolution, the solution pH was adjusted to pH 5.50 using 1 N HCl and ultrapure water was added to bring the final solution volume to 100 mL. To remove nucleases, diethylpyrocarbonate (DEPC) was added at 0.1% and incubated at room temperature overnight. The buffer solution was then autoclaved to remove DEPC. Polymer-siRNA complexes were formed by combining aqueous solutions of nanogels, siRNA, 10× RNAse-free PBS, and RNAse-free water to obtain desired concentrations.

Silencer® GAPDH siRNA, Quant-iT™ Ribogreen® RNA Assay Kit, and RNAse Free H₂O were purchased from Life Technologies (Carlsbad, Calif.). Free siRNA in solution was measured using the Ribogreen® assay according to manufacturer's instructions. Nanogel suspensions were diluted in RNAse free complexation buffer (pH 5.50). Concentrated siRNA was added to yield 500 ng ml⁻¹ RNA in a nanogel suspension at designated concentrations. Measurements of the free siRNA were taken after 60, 120, and 180 minute complexation periods.

Cell Culture.

General cell culture reagents and cell maintenance/passaging procedures are found in Section 5.2.3. In all cytometry studies, Caco-2 cells or RAW 264.7 cells were seeded to 6-well plates and allowed to grow to 80% confluency before use.

siRNA Delivery.

DyLight 647-labeled small interfering RNA (Sense: DY647-UAAGGCUAUGAAGAGAUACUU) was purchased from Thermo Scientific (Lafayette, Colo.). Cy3-labeled Silencer® Negative Control No. 1 siRNA was purchased from Life Technologies (Carlsbad, Calif.). Fluorescent nanogels, PDETB30-OG488 and PDESSB30-OG488 were synthesized and purified.

Concentrated suspensions (20×) of fluorescent nanogels (PDETB30-OG488 or PDESSB30-OG488), fluorescent siRNA (DY647-siRNA or Cy3-siRNA), or fluorescent nanogels and fluorescent siRNA were prepared to contain 0.5 mg mL⁻¹ nanogel, 26.5 μg mL⁻¹ (2000 nM) siRNA, 1× complexation buffer, and RNAse free H₂O. Control samples (nanogel or siRNA) were prepared in a similar fashion, replacing the volume of the absent component(s) with RNAse free H₂O.

To separate nanogel/siRNA complexes from complexation buffer, 4 vol equivalents of acetone was added to the suspension following the designated complexation period. The acetone serves to induce a polyelectrolyte-ionomer transition. Suspensions were centrifuged at 15,000 rpm for 5 min and supernatant was discarded. Residual solvent evaporated after 15 min in a laminar flow hood. Polymer/siRNA complexes were resuspended in the original complexation volume of RNAse free PBS at pH 7.40.

Following resuspension, 100 μL of nanogel/siRNA complexes at 500 μg ml⁻¹ in PBS were added to each test well to yield a final concentration of 25 μg ml⁻¹. Control wells received 100 μL PBS or 100 μL of the corresponding nanogel-only or siRNA-only solution. Nanogel exposure occurred for designated time points at 37° C. or 4° C. Following the exposure period, cells were rinsed 3×DPBS (with calcium and magnesium) and the media was replaced with 2 mL serum-free DMEM.

For Image Stream cytometry, Hoechst 33342 was added to each well for nuclear staining at a final concentration of 2.5 μg ml⁻¹. The nuclear staining process was completed for 30 min for RAW 264.7 cells and 45 min for Caco-2 cells at 37° C., 5% CO₂. Following Hoechst incubation, cells were rinsed 3× with DPBS (w/out calcium and magnesium). No nuclear stain was used in conventional flow cytometry experiments.

RAW 264.7 cells were isolated by replacing the final DPBS wash with 1 mL flow cytometry buffer and gently scraping the cells. Cell suspensions from each well were transferred to microfuge tubes and centrifuged for 5 min at 500×g. The supernatant was discarded and cell pellet re-suspended in flow cytometry buffer. Flow cytometry buffer was prepared by combining FBS, DPBS, and N₃Na to form 1% FBS and 0.1% N₃Na in DPBS.

Caco-2 cells were isolated by replacing the final DPBS wash with 500 μL 0.25% trypsin-EDTA and incubating at 37° C., 5% CO2 for 8 min. Trypsin was neutralized by adding 3 mL DMEM with 10% FBS and without phenol red. Cell suspensions were centrifuged for 5 min at 500×g. The supernatant was discarded and cell pellet re-suspended in flow cytometry buffer.

All cell suspensions were kept on ice until analysis with Image Stream Cytometry. Propidium iodide (PI) was used as a live/dead discriminator and was added to cell suspensions immediately before analysis at a final concentration of 1 μg mL⁻¹.

Flow Cytometry.

Efficiency of PDETB30- and PDESSB30-mediated Cy3-siRNA delivery was compared using a BD FACSCalibur (San Jose, Calif.) flow cytometer equipped with lasers at 488 nm and 635 nm. Fluorescent data were collected using FL-2 (570-600 nm, Cy3) and FL-3 (653-669 nm, PI). Dead or dying cells were identified with propidium iodide. Typically, 40,000 cells were collected per sample.

Image Stream Cytometry.

Analysis of uptake mechanisms and siRNA delivery was conducted using an Amnis Image Stream (Seattle, Wash.) imaging flow cytometer equipped with lasers at 405 nm, 488 nm, 658 nm, and 785 nm. Fluorescent data were collected using Channel 1 (430-505 nm, Hoechst), Channel 2 (505-595 nm, OG488), Channel 4 (595-660 nm, PI), Channel 5 (660-745 nm, DY647), and Channel 6 (745-800 nm, side scatter). Brightfield images were collected in Channel 3.

Cells were imaged with a 60× objective. Fluid velocity was set to a nominal value of 40 mm/sec. Fluorescent compensation matrices were constructed using Amnis IDEAS® software and verified manually for proper fit. At least 5,000 cells were collected for analysis. Dead cells (PI positive) were excluded from analysis. Out-of-focus cells were also excluded from further analysis by gating the Gradient RMS feature in IDEAS® software. This feature detects image sharpness by calculating large changes in pixel values across the brightfield image. Typically, cells with Gradient RMS value <40 were considered out of focus.

siRNA-Mediated Gene Silencing.

GAPDH Positive Control siRNA, KD Alert Assay Kits, and 10× Phosphate Buffered Saline (RNAse free) were purchased from Life Technologies (Carlsbad, Calif.). Caco-2 cells were seeded to tissue-culture treated 96-well plates at 2,500 cells/well and allowed equilibrate 24 hours before use. GAPDH siRNA was loaded into PDETB30 or PDESSB30 nanogels. Following 60 min incubation in complexation buffer, nanogel/siRNA complexes were precipitated through the addition of acetone and centrifuged at 15,000 rpm for 5 min. Supernatant was discarded and complexes were resuspended in RNAse free PBS. Prior to use, Caco-2 cells were washed 1× with PBS and media replaced with serum free DMEM. Concentrated (20×) nanogel/siRNA complexes or control suspensions were added to test wells and incubated at 37° C., 5% CO₂ for 60 min. Following the 60 min exposure period, cells were washed 3× with pre-warmed PBS and media replaced with complete DMEM. Cells were incubated at 37° C., 5% CO₂ prior to conducting the KD Alert gene silencing assay according the manufacturer's instructions. Care was taken to adjust the microplate reader sensitivity to remain within the GAPDH enzyme calibration curve established according to the manufacturer's instructions.

Statistical Analysis.

Statistical comparisons between experimental and control groups were made with two-tailed, unpaired, Student's t-test. Differences were accepted as statistically significant with p<0.05.

Results and Discussion

Synthesis of Disulfide Crosslinker

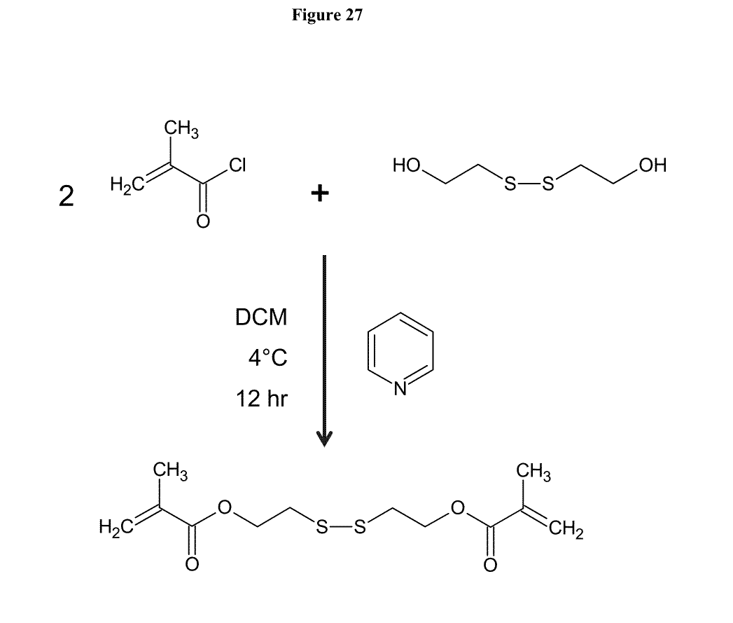

A homobifunctional crosslinker 2-bis-(2-methacryloyloxyethyl disulfide) was synthesized (FIG. 27) to endow responsive DEAEMA-based nanogels with a mechanism for biodegradation, namely reductive cleavage of the disulfide bonds. At the time of this study, this bifunctional linker was not commercially available. However, this crosslinker is now available commercially (with hydroquinone inhibitor) from Sigma-Aldrich (CAS No. 36837-97-5).

The synthesis and purification of 2-bis-(2-methacryloyloxyethyl disulfide) (SSXL) was successful; the reaction resulted in a molar yield of approximately 50%. The structure of SSXL was verified with ¹H-NMR. The spectra, (not shown), show peaks at δ=1.95 ppm (1H, H₂C═C—), 2.96 ppm (1H, H₂C═C—), 4.45 (2H, CH₂—CH₂—S—), 5.60 ppm (2H, —O—CH₂—CH₂—), and 6.14 ppm (3H, CH₃—C═). Analysis of the mass spectra (not shown) reveals a product of the expected molecular weight, 290 Da.

Degradable Nanogel Synthesis and Characterization

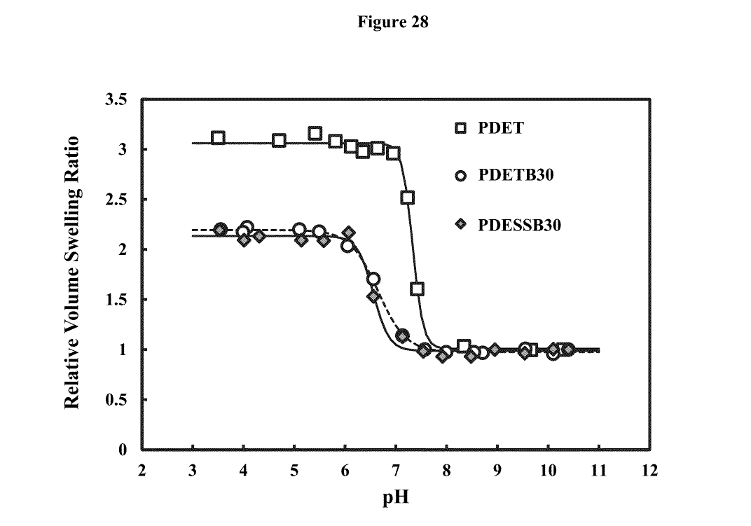

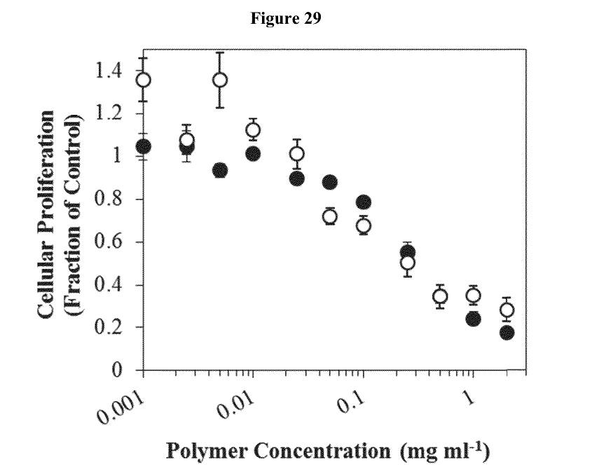

Disulfide-crosslinked nanogels containing an ionizable core of DEAEMA-co-TBMA and PEG corona were successfully synthesized via photoemulsion polymerization. Replacing the non-degradable linker TEGDMA (as used in PDET, PDETB30, etc) with SSXL had no identifiable change on physicochemical properties like pH-dependent swelling (FIG. 28), ξ-potential, and cytotoxicity to RAW 264.7 cells (FIG. 29).

As shown in FIG. 28, the critical swelling pH for PDESSB30 is 6.55. These nanogels have a z-average diameter of 96 nm at pH 8.5 and 126 nm at pH 6.0. The breadth of the volume phase transition is similar to PDETB30, occurring over 1.45 pH units. The value reported for PDETB30 (above) is 1.56 pH units. PDESBB30 nanogels exhibit a PdI of 0.12-0.15 throughout the volume phase transition.

The nanoscale dimensions of PDESSB30 were verified by TEM and the dry particle size was determined to be 50±17 nm. As tabulated in Table 5, this is quite similar to previous nanogel syntheses.

After confirming that size, swelling, and surface charge of the nanoscale hydrogels were unaffected by the change in crosslinker, studies were conducted to quantify the kinetics and extent of degradation in response to glutathione, a reductive tripeptide. Previous experiments served to establish a linear relationship between observed count rate (counts per second) and particle concentration (mg mL⁻¹) in dynamic light scattering experiments. This relationship was used to conduct a semi-quantitative, real-time measurement of degradation of SSXL-crosslinked nanoscale hydrogels in reductive aqueous suspension. The experiments were conducted using a Malvern ZetaSizer Nano ZS with the sample cell set to 37° C. and measurements collected approximately every 3 minutes. After a brief equilibration period, sample cuvettes were injected with PBS or aqueous glutathione to bring the final concentration to 1 mM or 10 mM glutathione in PBS.

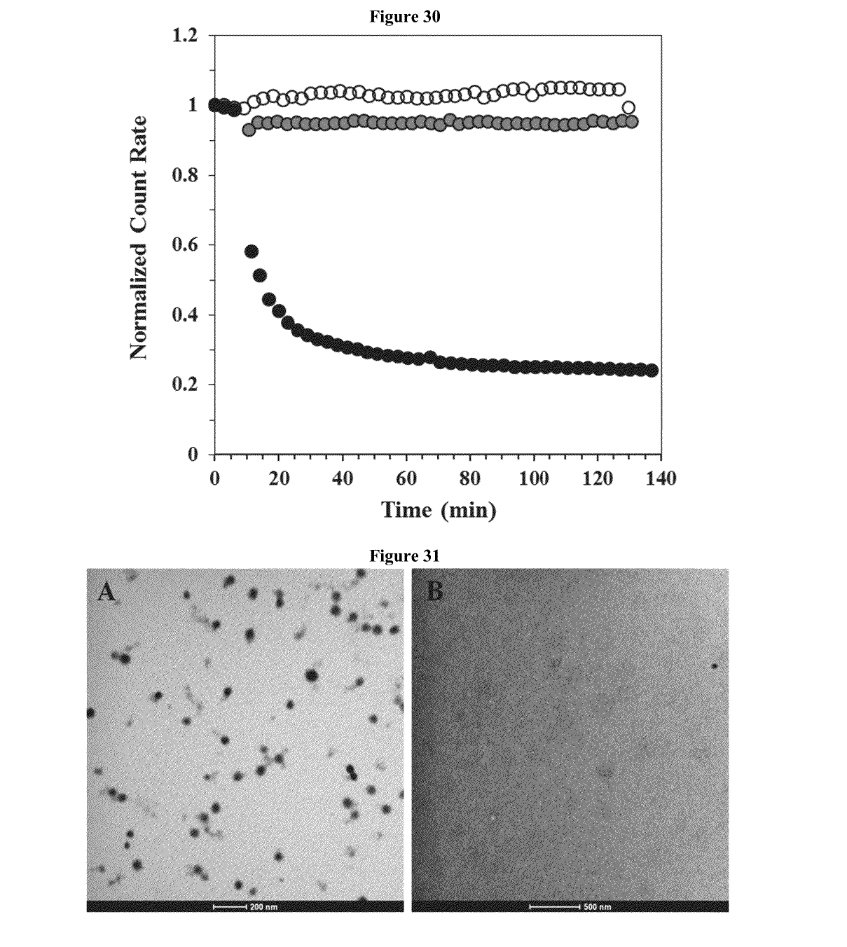

The degradation profiles, as seen in FIG. 30, offer insight on the apparent glutathione sensitivity and kinetics of polymer degradation. A reduction in observed count rate is taken as a reduction in particle concentration and is an indicator of glutathione-induced degradation. Interestingly, the concentration of glutathione required to induce significant polymer degradation lies between the average minimum (1 mM) and maximum (11 mM) concentrations of intracellular glutathione. This attribute is compelling because it gives SSXL-crosslinked nanoscale hydrogels the ability to remain intact in the extracellular milieu and degrade in the intracellular environment. These data indicate the time-scale for degradation is fairly rapid, on the order of minutes.

Upon exposure to 10 mM glutathione, nearly 50% of the PDESSB30 degradation exposed to 10 mM GSH occurs within 15 min. Nearly all degradation is completed by 60 minutes. Similar results for count rate degradation were observed for dithiothreitol (DTT), but are not shown here. For comparison, the observed count rate for PDESSB30 in PBS remained constant over the time-course of the experiment.

These observations were confirmed using Nanosight (Amesbury, UK) nanoparticle tracking analysis. At an identical glutathione:PDESSB30 to that employed in light scattering studies, exposure to 10 mM glutathione for 15 min at 37° C. caused a 52% reduction in the particle concentration from 33.2×10⁸ particles ml⁻¹ to 17.5×10⁸ particles ml⁻¹ (data not shown).

After 2 hours exposure to GSH, samples were inspected visually via TEM for evidence of degradation. As seen in FIG. 31, discrete particles were not detected at any region on the TEM grid, indicating near complete degradation of polymer networks.

This application of light scattering to monitor PDESSB30 degradation kinetics worked well for this initial application, though other methods may provide complementary insight into the degradation kinetics and extent of disulfide degradation. Analysis of degradation fragments using gel permeation chromatography (GPC) was problematic due to the high polysdispersity of the linear polymer chains and incomplete nanogel degradation at intermediate time points. Colorimetric methods, such as Ellman's assay, were not sufficiently sensitive to detect free sulfhydryl groups in degraded PDESSB30. The theoretical maximum (assuming 100% conversion) for sulfhydryl groups at 1 mg mL⁻¹ PDESSB30 in aqueous suspension is approximately 150 nM. Increasing the nanogel concentration beyond this concentration resulted in significant absorption due to solution turbidity.

Fluorescent Nanogel Synthesis

To enable visualization of nanogel subcellular localization in siRNA delivery experiments, a fluorescent version of the PDESSB30 nanogel was necessary. A primary amine-containing analogue of PDESSB30, termed PDESSB30f, was successfully synthesized and purified.

Oregon Green 488 (OG488), an amine reactive dye, was conjugated to primary amines in the nanogel core. Prior to the conjugation reaction, the primary amine content of PDESSB30f was determined to be 17.0±0.4 μmol g⁻¹, which represents a 11.5% incorporation efficiency. OG488 was subsequently added to PDESSB30f at 1:1 mol ratio of dye to amine. Following dialysis and lyophilization, the Oregon Green 488 functionalization was tested with fluorescence spectroscopy and the percent functionalization calculated with UV absorbance and comparison to an Oregon Green 488 standard curve.

The fluorescence emission (λₑₓ=465 nm) spectra of the labeled nanogel (PDESSB30-OG488) was determined. The fluorescent labeling was estimated at 16.9+0.3 μmol g⁻¹ using a standard curve of OG488 in PBS and at 19.5 μmol g⁻¹ using the absorbance at 496 nm and the OG-488 extinction coefficient (∈) of 70,000 L mol⁻¹ cm⁻¹, suggesting near 100% conjugation efficiency.

siRNA Loading Efficiency of pH-Responsive Nanogels

The ability of nanogels to encapsulate siRNA is an important determinant in their ability to deliver therapeutically relevant dosages of RNA to disease sites and initiate gene silencing. PDETB30 clearly displays the most desirable attributes for an endosomolytic drug delivery vehicle, but also contains fewer ionizable DEAEMA groups than PDET or PDETBA30. Previous work demonstrated that cationic polymer were able to bind DNA more efficiently with increasing cationic density. Thus, the RNA binding capacity was evaluated in a high-throughput fashion. RNA binding was evaluated as a function of nanogel composition, RNA:nanogel mass ratio, and complexation time to determine the loading efficiency of each nanogel.

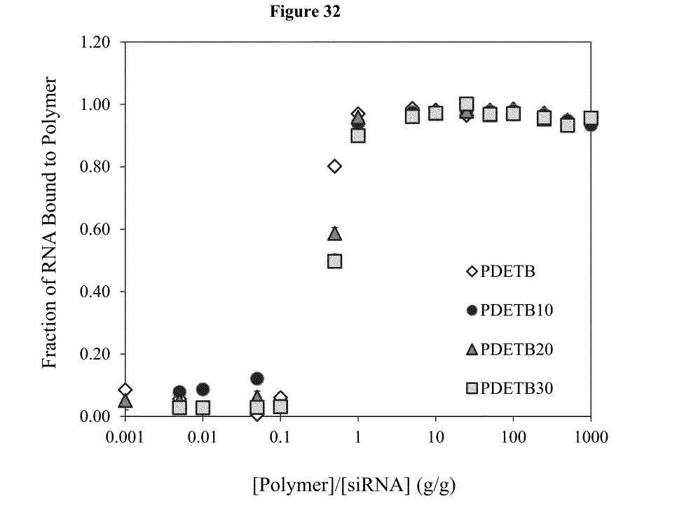

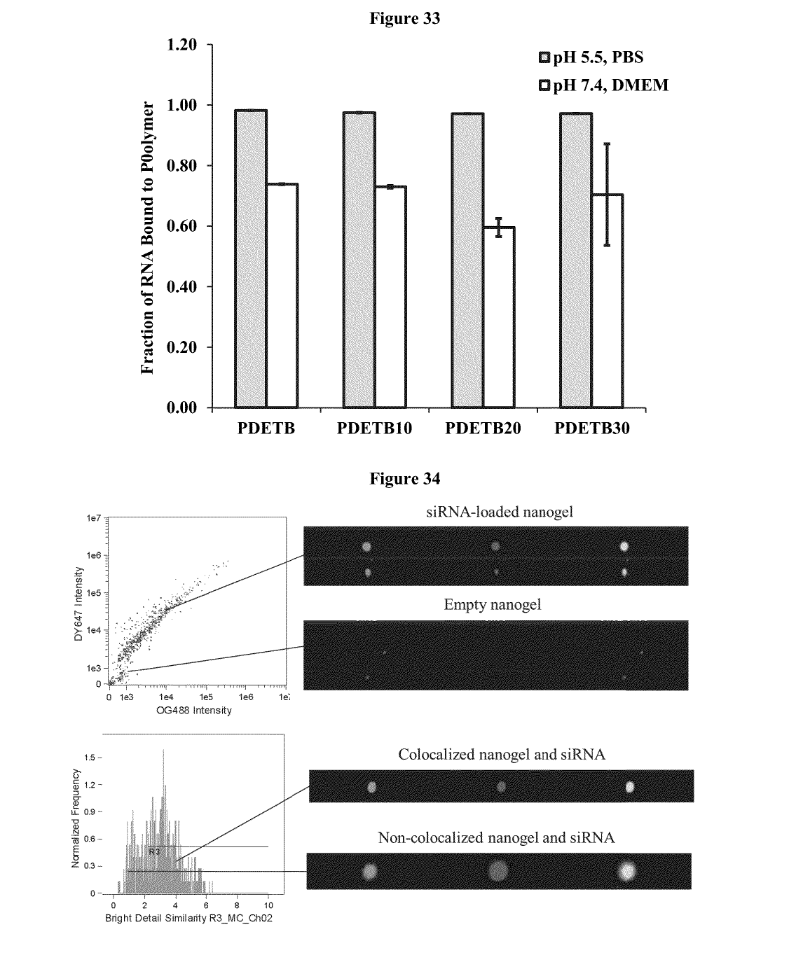

The fraction of free RNA (F_{f}) was calculated by taking the ratio of fluorescent intensity of sample solutions to fluorescent intensity of a polymer-free control RNA solution. Both measurements were corrected for background fluorescence and the fraction of bound RNA, F_{b}=1−F_{f}. These data for a 60 min complexation period are in FIG. 32. These results indicate that all formulations can efficiently bind free siRNA until a 1:1 mass ratio of polymer and siRNA and this binding is relatively independent of polymer composition. Nearly identical results were obtained for complexation periods of 120 and 180 min.

One limitation of this experimental technique is that it cannot distinguish the difference between RNA bound through surface adsorption or electrostatic encapsulation. Of these two, the latter is the more desirable as encapsulation and entrapment will afford better protection of RNA cargoes. As discussed previously, all polymer networks exhibit a positive surface charge at pH 5.50, so it is highly probable that anionic RNA can be adsorbed onto the particle surface through electrostatic interactions. Thus, this initial experiment likely overestimates the true RNA loading capacity of the nanogels.

To separate the effects of surface adsorption and electrostatic encapsulation, nanogels polymer and siRNA were allowed to complex in the acidic complexation buffer (PBS, pH 5.50) for 60 minutes and were subsequently transferred to 3× volume of serum free DMEM. By immersing the polymer/siRNA complexes in a more basic solution, the effective surface charge can be reduced from approximately 30 mV to nearly neutral (0±5 mV). This step change in surface charge serves to electrostatic interactions between nanogel surface and siRNA, permitting desorption of RNA from the surface.

According to the light scattering data presented in the above examples, this pH will completely or partially (depending on nanogel composition) collapse the network structure, serving to entrap RNA in the network core and limit diffusion out of the network. As seen in FIG. 34 for a 10:1 mass ratio of polymer to siRNA, approximately 70% of the RNA is retained in the bound state following immersion in DMEM. Sample loading efficiencies for each nanogel formulation are tabulated for 100:1 and 1:1 nanogel:siRNA ratios (g/g) in Table 7.

Qualitative evidence of nanogel/siRNA binding can be seen in FIG. 34. Complexes of PDETB30-OG488 and DY647-siRNA were visualized using Image Stream cytometry. As expected, the siRNA-loaded nanogels are too small to visualize with brightfield microscopy, but the fluorescent signal from PDETB30-OG488 and DY647-siRNA was visible. As seen in the DY647 vs. OG488 intensity plot in FIG. 34, nearly all PDETB30-OG488 nanogels contain DY647-siRNA. Analogous observations were made for complexes of PDESSB30-OG488 and DY647-siRNA.

TABLE 7
Loading efficiencies for nanogels and siRNA. Fraction of bound
RNA determined by Ribogreen assay following 60 min complexation
with nanogels. Data represent mean of triplicate samples ± s.d.

| | | Loading Efficiency | |
|---|---|---|---|
| Nanogel | Nanogel:siRNA (g/g) | pH 5.50 | pH 7.40 |
| PDET | 100 | 0.99 ± 0.003 | 0.72 ± 0.02 |
| PDETB10 | 100 | 0.98 ± 0.002 | 0.71 ± 0.01 |
| PDETB20 | 100 | 0.97 ± .0003 | 0.63 ± 0.01 |
| PDETB30 | 100 | 0.97 ± 0.001 | 0.67 ± 0.05 |
| PDET | 1 | 0.97 ± 0.001 | 0.58 ± 0.06 |
| PDETB10 | 1 | 0.94 ± 0.001 | 0.51 ± 0.09 |
| PDETB20 | 1 | 0.96 ± 0.002 | 0.50 ± 0.12 |
| PDETB30 | 1 | 0.90 ± 0.006 | 0.48 ± 0.06 |

Nanogel-Mediated Delivery of siRNA

Flow Cytometry

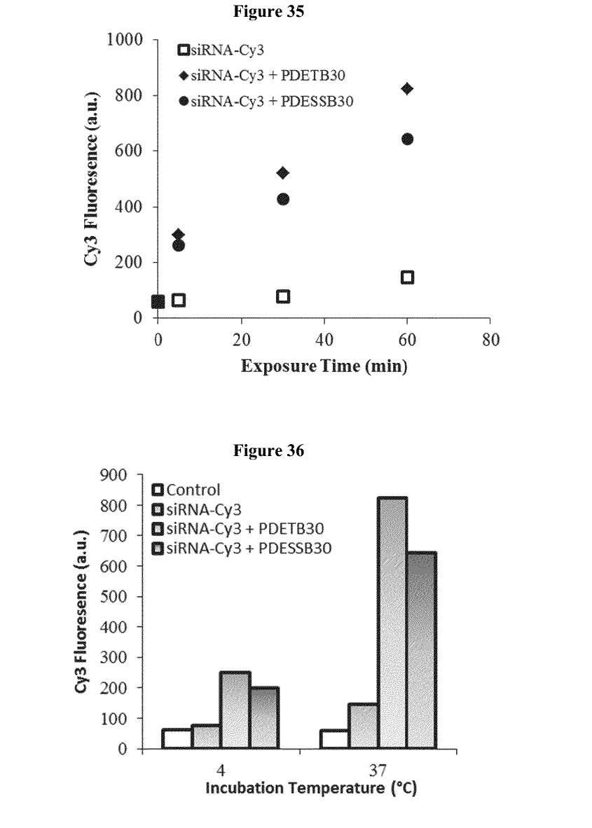

The ability of PDETB30, and its degradable analogue PDESSB30, to deliver fluorescent siRNA to Caco-2 cells was evaluated using flow cytometry. FIG. 35 shows the influence of exposure time on uptake of Cy3-siRNA. As expected, the naked siRNA is not able to efficiently enter Caco-2 cells. However, by complexation with PDETB30 or PDESSB30, the median fluorescence is increased by a factor of 5× after 5 min of exposure. Notably, siRNA delivery via PDETB30 and PDESSB30 result in a rapid increase in siRNA fluorescence from 0-5 min, followed by an approximately linear increase in median fluorescence from 5 min-60 min.

As discussed previous examples, the mechanism of internalization in Caco-2 cells is primarily macropinocytosis. This is an energy dependent internalization pathway and uptake of nanogel/Cy-3siRNA complexes is predictably inhibited by uptake at 4° C. (FIG. 36). Moreover, these data corroborate our observations that PDETB30 can be internalized through an energy-independent pathway. Following 60 min uptake at 4° C., cells exposed to PDETB30/Cy3-siRNA and PDESSB30/Cy3-siRNA exhibit greater median fluorescence than cells exposed to Cy3-siRNA alone.

These data suggest that PDETB30 and PDESSB30 are capable of delivery siRNA to Caco-2 cells. This work served as the basis for further study of the intracellular distribution of fluorescent nanogel/siRNA complexes and evaluation of the gene silencing activity of encapsulated siRNA.

Image Stream Cytometry

As described above, image Stream cytometry was used to simultaneously acquire statistical flow cytometry data and high-resolution fluorescent micrographs. Additionally, the imagine analysis capabilities of Image Stream cytometry permit the sorting and gating of events with particular image features, such as cellular internalization (vs. surface adsorption) or probe colocalizaition.

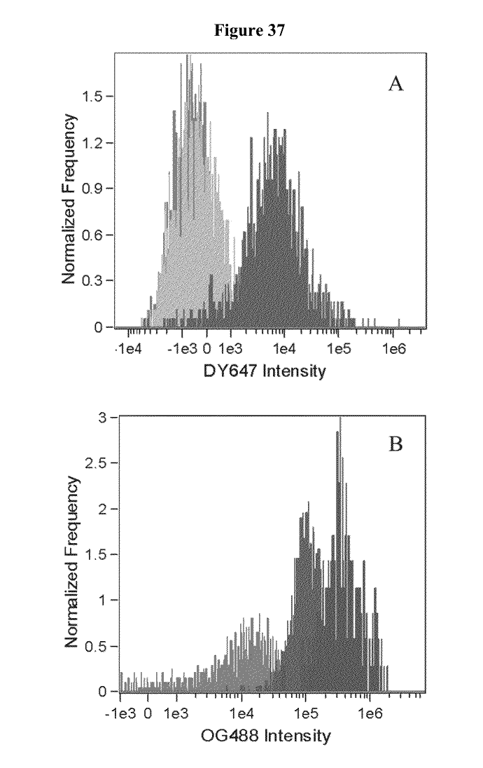

As shown in FIG. 37A, PDETB30-OG488 is an efficient delivery vehicle for DY647-siRNA in RAW macrophages. Both PDETB30-OG488 and PDESSB30-OG488 enhance the cytoplasmic fluorescence of DY647-siRNA relative to the siRNA only (blue histogram) and untreated control (gray histogram) cells. FIG. 37B shows the OG488 fluorescent intensity histogram in untreated (gray), PDETB30-OG488 (green), and PDETB30-OG488/DY647-siRNA (red) treated samples. Notably, the fluorescent signal in the cells treated with nanogels only is greater than that of cells treated with nanogels/siRNA. This suggests that the internalization of nanogel/siRNA complexes is less efficient than nanogels alone.

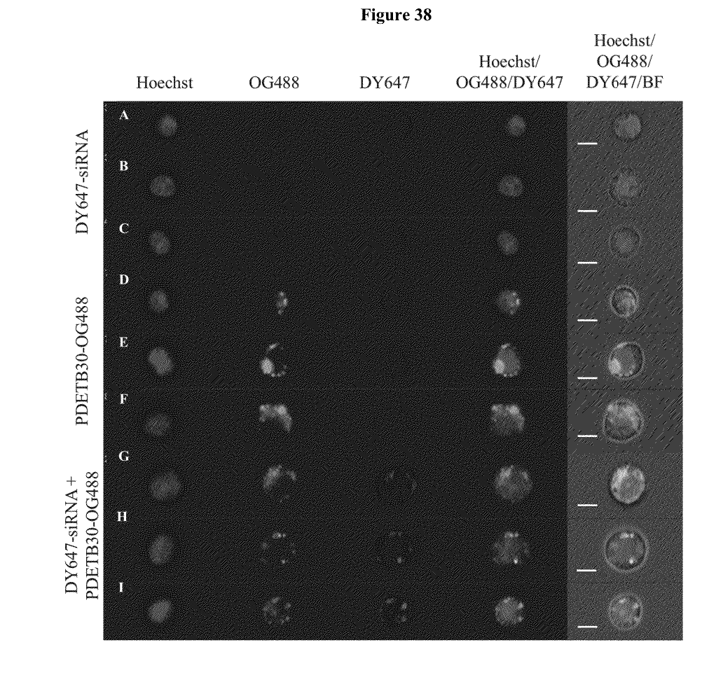

FIG. 38 shows representative fluorescent micrographs of RAW 264.7 cells. Cell nuclei are shown in blue (Hoechst), PDETB30-OG488 in green, and siRNA in red (DY647). Areas of nanogel/siRNA colocalizaition appear yellow on the fluorescent overlay. Panels A-C show representative images of cells exposed only to 100 nM DY647-siRNA for 60 min, panels D-F show representative images of cells exposed only to 25 μg mL⁻¹ PDETB30-OG488 for 60 min, and panels G-I show representative images of cells exposed 25 μg mL⁻¹ PDETB30-OG488 and 100 nM DY647-siRNA for 60 minutes. As expected due to its high MW (˜13 kDa) and negative charge, little to no internalization was observed by the naked siRNA. In panels G-I (PDETB30-OG488/DY647-siRNA), the siRNA staining pattern appears mostly diffuse and distributed near the cell membrane. Several bright, colocalized spots appear in panels H and I, suggesting some vesicular entrapment of polymer/siRNA complexes. Vesicular entrapment, and/or lysosomal accumulation, is expected in a portion of the nanogel/siRNA complexes due to the heterogeneous internalization pathways presented by RAW 264.7 cells.

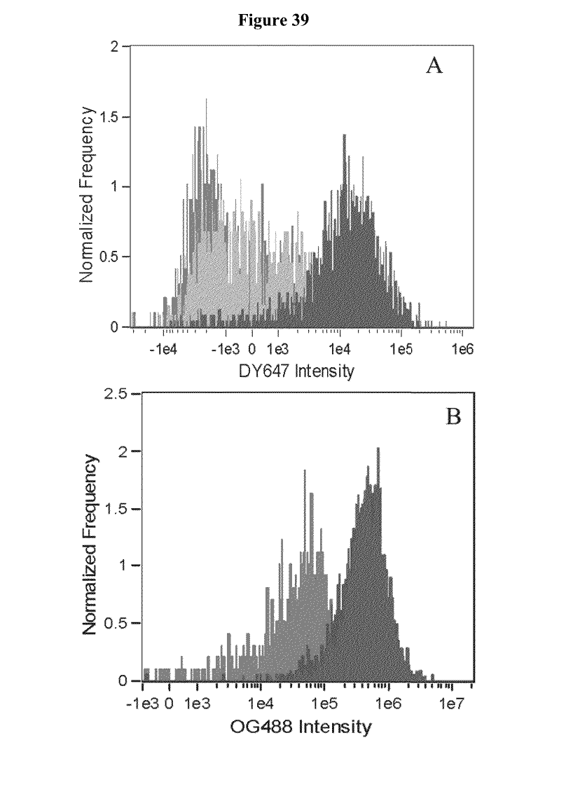

Similar observations were made for Caco-2 cells exposed to PDETB30/DY647-siRNA or DY647-siRNA under identical conditions. The fluorescent intensity histograms for DY64 and OG488 are shown in FIG. 39A and FIG. 39B, respectively. Comparing the median fluorescent values from FIG. 37 (RAW 264.7 cells) and FIG. 39 (Caco-2 cells) suggests a comparable level of nanogel and siRNA internalization in to these two cell types. The notable exception to this observation is the decreased PDETB30-OG488 intensity in RAW cells exposed to PDETB30-OG488/DY647-siRNA. This effect is not present in Caco-2 cells, which rely primarily on macropinocytosis to internalize PDETB30-OG488. RAW 264.7 cells uptake PDETB30-OG488 through a combination of clathrin-mediated endocytosis and macropinocytosis. This suggests that the clathrin-dependent uptake of nanogels is hampered by the presence of siRNA in the nanogel.

siRNA-Mediated Gene Silencing

Delivery efficacy of functional siRNA to model cells lines is an important parameter in the assessment of polymeric delivery systems. In these studies, GAPDH was chosen as the target gene for siRNA knockdown. GAPDH is a well-known housekeeping gene, ubiquitously expressed in nearly all cell types, and is involved in the reduction of NAD to NADH in the glycolysis pathway. Knockdown was assessed using a KDAlert™ GAPDH Assay Kit and monitoring the increase in fluorescence (em:520/ex:590) over a 4 minute period. This gene target was originally selected to facilitate a broad comparison of gene knockdown and transfection conditions for the cell types (Caco-2 and RAW 264.7) chosen for these studies.

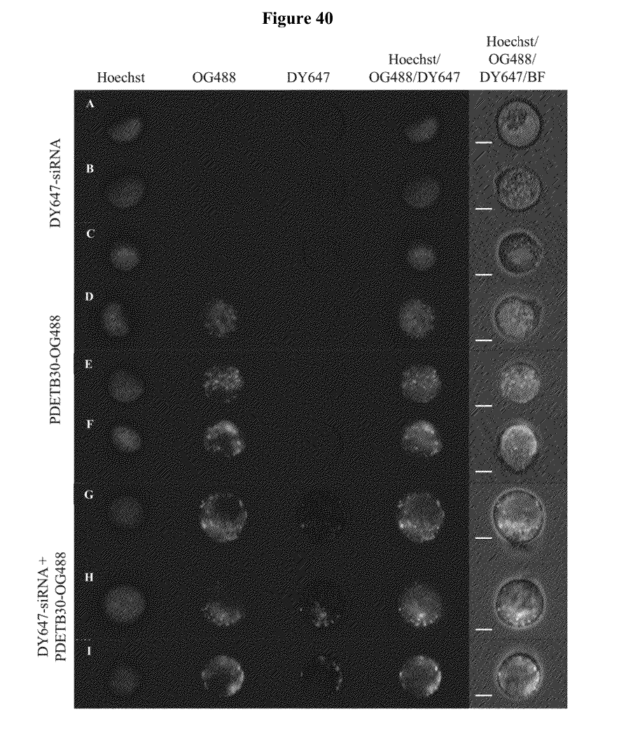

GAPDH knockdown, shown in FIG. 40, reveal that GAPDH siRNA delivered via PDETB30 induces a robust gene silencing effect, reducing GAPDH expression by 60-85%. This knockdown effect occurred at multiple polymer:siRNA ratios ranging from 8:1-1000:1.

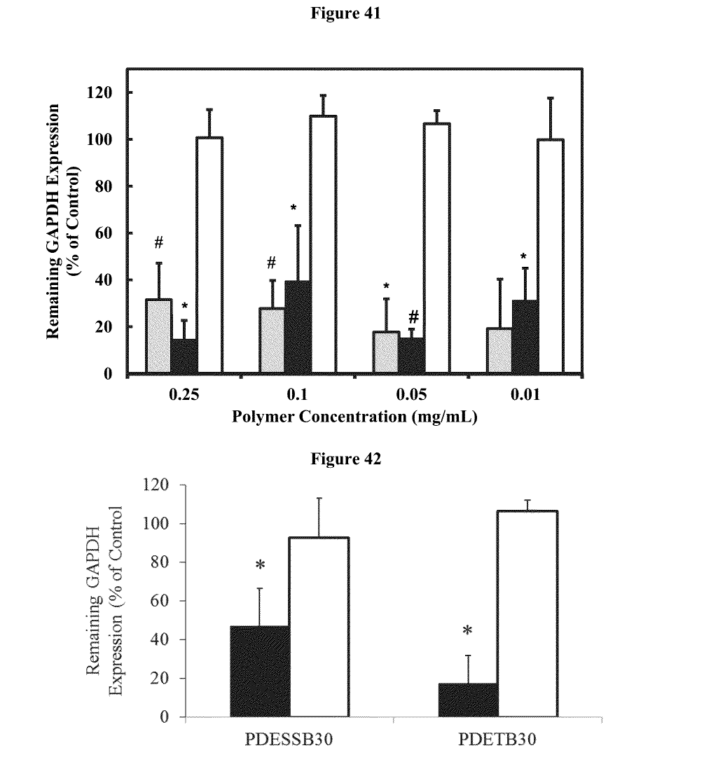

FIGS. 41 and 42 compares the siRNA-mediated gene silencing in Caco-2 cells treated with PDETB30/siRNA or PDESSB30/siRNA at a 200:1 nanogel:siRNA ratio. Both nanogel formulations are capable of delivering functional siRNA. Knockdown efficiency was 53% for cells treated with PDESSB30/siRNA and 83% for cells treated with PDETB30/siRNA. Based on flow cytometry data in FIG. 35, Caco-2 cells treated with PDETB30/siRNA exhibited characteristically higher siRNA fluorescence than did cells treated with PDESSB30/siRNA. Therefore, the superior knockdown efficiency of PDETB30/siRNA relative to PDESSB30/siRNA is likely due to increased siRNA delivery efficiency by the former combination.

While GAPDH siRNA and KD Alert assays were suitable for initial studies of gene silencing in Caco-2 cells, this assay did not transfer well to RAW 264.7 macrophages. The linear range for detecting GAPDH enzyme activity can typically accommodate approximately 2,000-10,000 cells/well. In Caco-2 cells (doubling time ˜60 h), the cell density at the time of assay (˜48 h after transfection) is expected to be 1-2× the seeding density. In contrast, RAW 264.7 cells grow much more rapidly, with a doubling time ˜15 h. Therefore, RAW 264.7 cells will undergo 3-4 doubling cycles between transfection and assay. Thus, RAW 264.7 cells were typically seeded at a low density (1,000 cells/well in 96-well plates) for these studies. Any variations in the cell seeding density were then amplified by successive rounds of RAW division. This becomes problematic because the KD Alert relies on comparison of experimental wells to external control wells. In practice, high variability in the RAW cell density at assay time limited the utility of the KD Alert assay kit in evaluating any PDETB30/siRNA-mediated gene silencing in RAW cells. Analysis methods that provide an internal control, such as qPCR, are better suited for evaluation of gene silencing.

### CONCLUSIONS

A disulfide crosslinker was synthesized to allow degradation of pH-responsive nanogels in reductive environments. This crosslinker was incorporated into responsive nanogels, termed PDESSB30, with little to no changes on physicochemical properties including critical swelling pH, nanogel size, or in vitro biocompatibility. These nanogels degrade within minutes upon exposure to physiological levels of glutathione as determined by light scattering and electron microscopy. Analysis of cellular internalization demonstrated efficient uptake of siRNA delivered via degradable (PDESSB30) and non-degradable (PDETB30) nanogels. Moreover, both PDESSB30 and PDETB30 are capable of delivering functional siRNA to Caco-2 cells, achieving gene silencing of 47% and 83%, respectively. The combination of attractive physicochemical properties and siRNA delivery efficiency make PDETB30 and PDESSB30 attractive as therapeutic siRNA delivery systems.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. A pH responsive polycationic hydrogel comprising a cationic monomer, a hydrophobic moiety, and a crosslinker.

2. The composition of claim 1, wherein the amount of cationic monomer and hydrophobic moiety is present in a ratio of from about 20% to about 50%.

3. The composition of claim 1, wherein the cationic monomer is from 50 to 80 mol %, the hydrophobic moiety is from 20 to 50 mol %, and the crosslinker is from 0.5 to 5 mol %.

4. The composition of claim 1, wherein the hydrogel is greater than 80% cytocompatible at 100 ug/mL.

5. The composition of claim 1, wherein the hydrogel has a positive surface charge at about pH 7.4.

6. The composition of claim 1, wherein the hydrogel has a collapsed structure at about pH 7.4.

7. The composition of claim 1, further comprising a plurality of poly(ethylene glycol) polymers covalently attached to the hydrogel.

8. The composition of claim 1, further comprising a plurality of polyoxazoline polymers covalently attached to the hydrogel.

9. The composition of claim 1, further comprising a plurality of poly(ethylene glycol) or polyoxazoline polymers or both covalently attached to and at least partially disposed on an exterior surface of the hydrogel.

10. The composition of claim 1 wherein the cationic monomer is 2-(diethylamino) ethyl methacrylate (DEAEMA).

11. The composition of claim 1 wherein the cationic monomer is 2-(tert-butylamino)ethyl methacrylate (BAEMA).

12. The composition of claim 1 wherein the cationic monomer is a tertiary amino methacrylate, a dimethyl amino ethyl methacrolyate, a diethyl amino ethyl methacrolyate, a diisopropyl amino ethyl methacrolyate, a morpholino ethyl methacrylate, a polylysine methacrylate, or a combination thereof.

13. The composition of claim 1 wherein the hydrophobic moiety is tert-butyl methacrylate (BMA).

14. The composition of claim 1 wherein the hydrophobic moiety is a reactive methacrylate or acrylates.

15. The composition of claim 1 wherein the hydrophobic moiety is an aliphatic (meth)acrylate, a propyl (meth)acrylate, a tert-butyl (meth)acrylate, a 2-(tert-Butylamino)ethyl methacrylate, a n-butyl (meth)acrylate, a phenyl (meth)acrylate, an iso-butyl (meth)acrylate, a hexyl (meth)acrylate, an iso-decyl (meth)acrylate, a lauryl (meth)acrylate, or a combination thereof.

16. The composition of claim 1 wherein the crosslinker is degradable.

17. The composition of claim 1 wherein the crosslinker is a homobifunctional methacrylate.

18. The composition of claim 1 wherein the crosslinker is ethylene glycol dimethacrylate (EGDMA), tetra(ethylene glycol) dimethacrylate (TEGDMA), poly(ethylene glycol) dimethacrylate (PEGDMA), bis(2-methacryloyloxyethyl) disulfide, or a combination thereof.

19. The composition of claim 1, further comprising an anionic therapeutic disposed within the hydrogel.

20. The composition of claim 1, further comprising siRNA disposed within the hydrogel.

21. The composition of claim 1, wherein the hydrogel has a z-average particle size diameter of from about 20 nm to about 200 nm.

22. The composition of claim 1 wherein the polycationic network has responded to a change in pH.

23. A pharmaceutical formulation comprising a composition according to claim 1.

24. A method comprising:
providing at a pH of less than or equal to about 6.5a pH responsive polycationic hydrogel comprising a cationic monomer, a hydrophobic moiety, and a crosslinker;
introducing the pH responsive polycationic hydrogel to an environment having a pH of about greater than or equal to about 7.

25. The method of claim 24, wherein the cationic monomer is from 50 to 80 mol %, the hydrophobic moiety is from 20 to 50 mol %, and the crosslinker is from 0.5 to 5 mol %.

26. The method of claim 24, wherein the pH responsive polycationic hydrogel further comprises an anionic therapeutic disposed within the hydrogel.

27. The method of claim 24, wherein the pH responsive polycationic hydrogel further comprises siRNA disposed within the hydrogel.
